(19) United States
(12) Patent Application Publication
(10) Pub. No.: US 2013/0344501 A1
(43) Pub. Date: Dec. 26, 2013

(54) METHODS FOR PRODUCING THREE-DIMENSIONAL PHYSIOLOGICALLY RELEVANT IMMUNE TISSUE SYSTEMS UNDER LOW FLUID SHEAR CONDITIONS

(71) Applicants: Cheryl Anne Nickerson, Phoenix, AZ (US); Aurelie Crabbe, Phoenix, AZ (US); Shameema Sarker, 85249, AZ (US)

(72) Inventors: Cheryl Anne Nickerson, Phoenix, AZ (US); Aurelie Crabbe, Phoenix, AZ (US); Shameema Sarker, 85249, AZ (US)

(21) Appl. No.: 13/878,992

(22) PCT Filed: Oct. 31, 2011
(86) PCT No.: PCT/US11/58625

§ 371 Date: Sep. 6, 2013

Related U.S. Application Data
(60) Provisional application No. 61/408,528, filed on Oct. 29, 2010.

(51) Int. Cl. C12N 5/00

(52) U.S. Cl. CPC C12N 5/0062
USPC 435/7.1; 435/373; 435/366; 435/372; 435/29; 435/347

(57) ABSTRACT

Methods of producing a three-dimensional, physiologically relevant immune tissue system, including culturing an immune cell and at least one other cell type separately; placing immune cell and the at least one other cell type in a low fluid shear environment for a time period; and co-culturing the cells under conditions selected to produce a three-dimensional immune tissue system with physiologically relevant characteristics.

This application claims the benefit of the filing date of U.S. Provisional Patent Application No. 61/408, 528, filed Oct. 29, 2010, the contents of which are hereby incorporated by reference.

### STATEMENT REGARDING FEDERALLY SPONSORED RESEARCH OR DEVELOPMENT

The present invention was made with United States government support under Grant No. HHS-NIH-NIMH R21MH080702 awarded by the National Institutes of Health (NIH) and NCC2-1362, NNJ04HF75F, and NNJ06HE92 awarded by the National Aeronautics and Space Administration (NASA). The United States government may have certain rights in this invention.

### FIELD OF THE INVENTION

The present invention relates generally to the fields of microbiology, infectious disease, immunology, cell biology, toxicology, cancer, environmental microbiology, bioengineering, biotechnology, vaccine/adjuvant/therapeutic and drug development.

### BACKGROUND

Conventional two-dimensional (2-D) cell culture involves growing cells as monolayers on solid, impermeable surfaces (plastic or glass) or in uniform suspension. However, this “flat biology” 2-D approach, in which key phenotypic and functional characteristics are lost, often does not accurately predict in vivo tissue responses. (See, e.g., Bissell et al., Curr. Opin. Cell Biol. 15, 753-762 (2003)).

One key reason for the loss of differentiation that occurs in monolayers is the dissociation from the native in vivo three-dimensional structure to 2-D propagation on flat, impermeable substrates in vitro, which also prevents cells from responding to chemical and molecular gradients in three dimensions (reflecting the apical, basal and lateral cell surfaces) (Abbott, A. Nature 424, 870-872 (2003); Freshney, R. Culture of Animal Cells: a Manual of Basic Technique (Wiley-Liss, New York, 2000); Schmeichel, K. L. & Bissell, et al., J. Cell Sci. 116, 2377-2388 (2003); O'Brien, L. E., et al., Nature Rev. Mol. Cell. Biol. 3, 531-537 (2002)). Because 2-D monolayers lack the complexity, and often the physiological relevance, of the tissues that are encountered by a pathogen during the natural course of infection in vivo, they are often unfaithful predictors of the infection process.

Thus, to overcome some of the inherent limitations associated with 2-D monolayers, as well as the high cost, availability and variability of animal models, a need exists for three-dimensional tissue systems. A three-dimensional tissue system can help bridge the gap between cell-based discovery research and animal models for studying both host-pathogen interactions and disease progression, as well as for the development of novel drugs and therapeutics.

One challenge in developing physiologically relevant tissue systems, such as an immunocompetent tissue system, is in recapitulating the dynamic integrated three-dimensional network of lymphoid cells, innate immune cells, epithelial cells, effector molecules, and cellular microenvironments, all of which are vital for host protection and defense. Conventional cell culture technologies, including alternative three-dimensional tissue culture systems, do not provide the comprehensive structural and functional aspects that are required to produce physiologically relevant equivalents of the immune system.

### SUMMARY

This disclosure describes methods for producing three-dimensional, physiologically relevant immune tissue systems under low fluid shear conditions.

In one aspect, a method of producing a three-dimensional, physiologically relevant immune tissue system is disclosed. The method includes a) introducing an immune cell and at least one other cell type into a low fluid shear environment; and b) co-culturing the immune cell and the at least one other cell type under conditions selected to produce a three-dimensional immune tissue system with one or more physiologically relevant characteristics.

In one or more embodiments, the one or more physiologically relevant characteristics are selected from the group consisting of one or more differentiated and functional cells, assembly into relevant three-dimensional aggregates, production of extracellular matrix components, and physiologically relevant cell type ratios.

In one or more embodiments, the immune cells are selected from the group consisting of monocytes, astrocytes, neuronal cells, macrophages, dendritic cells, B cells, T cells, natural killer cells, basophils, eosinophils, and neutrophils from healthy and/or diseased subjects.

In one or more embodiments, the immune cells are astrocytes and neuronal cells.

In one or more embodiments, the immune cells further comprise monocytes.

In one or more embodiments, the method further includes culturing the immune cell and/or the at least one other cell type in a monolayer before placing in the low fluid shear environment.

In one or more embodiments, the method further includes developing the immune cell and/or at least one other cell type into three-dimensional cells before placing in the low fluid shear environment.

In one or more embodiments, the method includes first placing the immune cell in the low fluid shear environment, first placing the at least one other cell type in the low fluid shear environment, or placing the immune cell and the at least one other cell type simultaneously in the low fluid shear environment.

In one or more embodiments, the method includes developing the cells into three-dimensional cells on a scaffold. In one or more embodiments, the scaffold is made of microcarrier beads.

In one or more embodiments, the at least one other cell type is an immune or epithelial cell.

In one or more embodiments, the one or more physiologically relevant characteristics are selected from the group consisting of a differentiated epithelium, one or more functional macrophage-like cells, a localization of macrophage-like cells on or beneath the epithelial surface, production of one or more extracellular matrix components, and a physiologically relevant macrophage-to-epithelial cell ratio.

In one or more embodiments, the physiologically relevant macrophage-to-epithelial cell ratio ranges from about 1:30 to about 1:40.

In one or more embodiments, the epithelial cells are selected from the group consisting of alveolar, bronchial, small intestinal, large intestinal, cervical, urogenital, gastrointestinal tract, respiratory tract, and vaginal epithelial cells from healthy and/or diseased subjects and vaginal epithelial cells from healthy and/or diseased subjects.

In one or more embodiments, the epithelial cells and the immune cells are derived from human cell lines.

In one or more embodiments, the epithelial cells are small intestinal epithelial cells, and the immune cells are monocytes.

In one or more embodiments, the epithelial cells are large intestinal epithelial cells, and the immune cells are monocytes.

In one or more embodiments, the immune cell is a monocyte, and the at least one other cell type is an alveolar epithelial cell.

In one or more embodiments, the method further includes culturing the alveolar epithelial cells in a monolayer and developing the alveolar epithelial cells into three-dimensional cells in the low fluid shear environment.

In one or more embodiments, the ratio of the monocytes to the three-dimensional alveolar epithelial cells ranges from about 1:100 to about 100:1.

In one or more embodiments, the low fluid shear conditions ranges from about 0 dynes/cm² to about 10.0 dynes/cm.²

In one or more embodiments, the time period of step b) ranges from about 1 day to about 40 days.

In one or more embodiments, the low fluid shear environment is provided by one or more bioreactors.

In one or more embodiments, the bioreactor is a rotating wall vessel (RWV).

In one or more embodiments, the bioreactor has a rotation speed that ranges from about 10 rpm to about 30 rpm.

In one or more embodiments, the RWV is a slow transfer lateral vessel (STLV).

In one or more embodiments, the RWV is a high-aspect rotating vessel (HARV).

In one or more embodiments, the low fluid shear environment is a spaceflight environment.

In one or more embodiments, the conditions appropriate for producing a three-dimensional immune tissue system with physiologically relevant characteristics are selected from the group consisting of appropriate culture medium, temperature, oxygen level, pH, composition of the extracellular matrix, and time in the low fluid shear environment.

In one or more embodiments, the culture medium is GTSF-2.

In one or more embodiments, the time in the low fluid shear environment ranges from about 24 hours to about 1 year.

In one or more embodiments, the method further includes conducting one or more biochemical analyses to determine that the three-dimensional tissue system has one or more physiologically relevant characteristics.

In another aspect, the disclosure includes a kit for producing a three-dimensional, physiologically relevant tissue system, comprising an immune cell line and at least one other cell line; and informational material for producing a three-dimensional, physiologically relevant tissue system.

### BRIEF DESCRIPTION OF THE DRAWINGS

The foregoing and other objects of the present invention, the various features thereof, as well as the invention itself, may be more fully understood from the following description, when read together with the accompanying drawings. The following drawings are presented for the purpose of illustration only, and are not intended to be limiting.

FIGS. 1A-D shows immunohistochemical profiling of monocytes/macrophages in the three-dimensional A549-U937 co-culture (72 hour co-cultivation).

FIGS. 2A-B shows ICAM-1 expression and phagocytosis activity of three-dimensional A549-U937 co-cultures, undifferentiated monocytes, and PMA-differentiated macrophages.

FIG. 3 shows the concentration of 3-oxo-C₁₂ HSL as a function of time after exposure to three-dimensional A549, three-dimensional A549-U937, and U937 cultures.

FIG. 4 shows the ratio of the ICAM-1 signal intensity of co-cultured A549 cells and U937 cells exposed to 3-oxo-C₁₂ HSL, to the ICAM-1 signal intensity of their respective DMSO solvent controls.

FIG. 5 shows one operation of the rotating wall vessel (RWV) technology.

FIGS. 6A-B shows the immunohistochemical profiling of epithelial differentiation markers in three-dimensional A549, three-dimensional A549-U937, and A549 monolayer cultures.

FIG. 7 shows the viability of U937 cells as a function of time following exposure to 0 μM (DMSO control) and 100 μM 3-oxo-C₁₂ HSL. Standard deviations for U937 exposed to 0 μM and 100 μM 3-oxo-C₁₂ HSL were lower than 1% and 2% respectively and are therefore not visible on the chart (N=2).

FIG. 8 shows the standard curve relating 3-oxo-C₁₂ HSL concentration to the diameter of the green fluorescent protein (GFP) fluorescent circle of the indicator strain on the overlay plate assay. Error bars represent the mean±standard deviation (N=2).

FIGS. 9A-B shows the viability of U937 cells following exposure to 100 μM 3-oxo-C₁₂ HSL or DMSO, when cultured alone and with three-dimensional A549 cells.

FIGS. 10A-K shows generation of an extracellular matrix (ECM) by monocytes through light microscopy, SEM, and confocal images of U937 cells attached to microcarrier beads and differentiating over a period of time in a bioreactor.

FIG. 11 are light microscopy images of U937 and HT29 cells added simultaneously to microcarrier beads in the bioreactor.

FIGS. 12A-J are light microscopy images of the kinetics of U937 cell attachment to beads (12A-B), 3-D aggregate formation by colon (HT29) and monocytic (U937) cells grown as 3-D co-cultures (12C-D), 3-D co-cultures (12E-H), and confocal images of 3-D co-cultures showing expression of CD45 and CD54 monocyte/macrophage markers (12I-J).

FIG. 13 are light microscopy, SEM, and confocal images of kinetics of 3-D aggregate formation by colon (HT29) and monocytic (U937) cells grown as 3-D co-cultures, SEM images of U937 cells grown for 24 hours on beads, HT29 cells on beads (second panel), 3-D co-cultures of HT29 and U937 cells (third panel) and comparison of CD54 (ICAM-1) expression in 3D HT29 monotypic culture vs. co-culture (fourth panel). The images are results from optimizing different growth conditions.

FIG. 14 shows light microscopy images showing kinetics of 3-D aggregate formation by colon (HT29) cells (first panel), 3-D HT29 and U937 co-cultures (second panel) and U937 bound to beads in the presence of PMA (third panel). The images are results from optimizing different growth conditions.

FIG. 15 shows immunohistochemical profiling of colon (HT29) cells and PMA differentiated macrophages (U937) grown as 3-D co-cultures (FIGS. B, D, F, G-L), and 3-D monotypic cultures (FIGS. A, C and E). Confocal images of cultures from various timepoints were stained with antibodies against epithelial differentiation markers MUC5AC (FIGS. A and B), ZO-1 (FIGS. C, D, E, F), and monocyte/macrophage markers CD45 (FIGS. G, I, J, L), and CD54 (FIG. K). Phagocytosis of 2 mm fluorescent beads by the 3-D HT29-U937 (PMA-differentiated) co-culture are shown (FIGS. H-J).

FIG. 16 shows immunohistochemical profiling showing phagocytosis of fluorescent beads by PMA differentiated macrophages (U937) grown as co-cultures with HT29 colonic epithelial cells. Blue—DAPI, shown as 3 (nuclear staining); yellow fluorescent beads, shown as 5.

FIG. 17 shows immunohistochemical profiling of small intestinal (Int-407) epithelial cells and PMA differentiated macrophages (U937) grown as 3-D co-cultures (FIGS. 17B and C), and 3-D monotypic cultures (FIG. 17A).

FIG. 18. Adherence, Invasion and/or Intracellular growth profiles of Salmonella Typhimurium and Salmonella Typhi in HT-29 monolayers and 3-D HT29 monotypic and co-cultures. Adherence/Invasion and/or Intracellular growth of wildtype Salmonella, Ty2 Typhi and the clinical isolate ISP1820, were measured at 1 hpi, 3 hpi, 24 hpi. and 48 hpi. For all cell culture models, HT-29 cells were infected at an m.o.i. of 10 and lysed at each indicated time point and plated to enumerate bacterial CFU.

FIG. 19 shows light microscopy images of kinetics of 3-D aggregate formation by neuronal (SH-SY5Y) and astrocyte (HTB-14) cells grown as 3-D co-cultures (first panel), 3-D neuronal cells (second panel) and 3-D astrocytes (third panel).

FIGS. 20A-B shows immunohistochemical profiling of neuronal (SH-SY5Y) and astrocyte (HTB-14) cells grown as 3-D co-cultures (A-D), 3-D monotypic cultures (E-F neuronal; I-L astrocytes), or monolayers (M-P neuronal; Q-T astrocytes). Confocal images of cultures from various timepoints were stained with antibodies against Tubulin (A,E,I,M,Q), GFAP (B,F,J,N, R), MAP2 (C,G,K,O,S), and NeuN (D,H,L,P,T).

FIG. 21 shows immunohistochemical profiling of neuronal (SH-SY5Y) and astrocyte (HTB-14/U87-MG) cells grown as 3-D co-cultures (A,C, F), 3-D monotypic cultures (D, neuronal; B, E astrocytes). Confocal images of cultures from various timepoints were stained with antibodies against Tubulin (B, C), GFAP (A), and NeuN (D-F).

FIG. 22 shows immunohistochemical profiling of astrocyte (HTB-14/U87-MG) cells grown as 3-D aggregates. Confocal images of cultures from Day 23 were stained with antibodies against Tubulin (A), GFAP (B), MAP-2 (C) Glutamine synthetase (D) and dual staining with GFAP and MAP-2 (E) and GFAP and Tubulin.

FIG. 23 shows immunohistochemical profiling of neuronal (SH-SY5Y) astrocyte (HTB-14) and monocytic cells (THP-1) grown as 3-D co-cultures (A-F) in a collagen coated ECM vs. laminin coated ECM on microcarrier beads. Confocal images of cultures from Day 14 were stained with antibodies against MAP-2 (A), GFAP (B and E), Tubulin (C and F), and NeuN (D).

FIG. 24 shows immunohistochemical profiling of neuronal (SH-SY5Y) and astrocyte (HTB-14/U87-MG) and acute monocytic leukemia cells (THP-1) grown as 3-D co-cultures (A-C). Confocal images of cultures from Day 14 were stained with antibodies against CD83 (A, C) and CD68 (B). Blue—DAPI, shown as 3 (nuclear staining); Green, shown as 2 (Cd83, CD68).

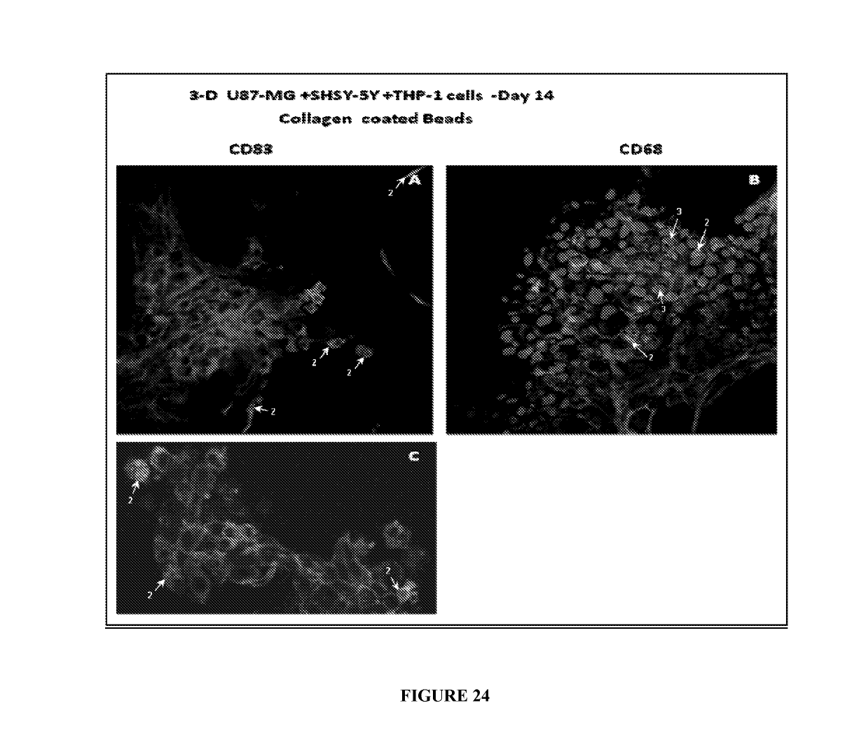

FIG. 25 shows a three-dimensional A549-U937 co-culture model infected with Pseudomonas aeruginosa PAO1. Arrow points out uptake of bacteria by phagocytic cell (image was derived from a movie where phagocytosis could be observed).

FIGS. 26B, D, and F shows immunohistochemical profiling of colon (HT29) cells and PMA-differentiated macrophages (U937) grown as three-dimensional co-cultures. FIGS. 26A, C, and E show three-dimensional 3-D monotypic cultures.

FIG. 27 shows scanning electron microscopy (FIG. 27A) and light microscopic (FIG. 27B) images of A549 monotypic and A549-U937 co-cultures uninfected (3 days of co-cultivation) and infected with P. aeruginosa PAO1.

FIG. 28 shows localization of P. aeruginosa PAO1 in the infected A549-U937 co-culture. Bacteria do not invade host cells (FIG. 28A) and remain in the epithelial mucus layer (FIG. 28B).

FIG. 29 shows mean ICAM-1 fluorescence in A549 monotypic and A549-U937 co-cultures before and after 20 hours infection with P. aeruginosa PAO1.

FIG. 30 shows cytokine concentrations of 3-D A549 monotypic and A549-U937 co-cultures before and after exposure to P. aeruginosa PAO1.

### DETAILED DESCRIPTION

### General

All patents, patent applications and publications cited herein are hereby incorporated by reference in their entirety to more fully describe the state of the art as known to those skilled therein as of the date of the invention described herein.

The disclosure is based, in part, on the discovery of methods for producing three-dimensional, physiologically relevant immune tissue systems under low fluid shear conditions. The disclosure also relates to methods of assessing disease pathogenesis and disease treatment using three-dimensional, physiologically relevant immune tissue systems, such as related to, for example, infectious disease, cancer, inflammation, chemosensitivity, toxicology, reactogenicity, vaccine/adjuvant/therapeutic and drug design, development and screening, immunogenicity, and tissue homeostasis and transition to disease. The disclosure also relates to kits for producing three-dimensional, physiologically relevant immune tissue systems under low fluid shear conditions.

The present disclosure presents methods that improve upon conventional cell culture methods and animal models to better predict in vivo human responses to infectious pathogens, toxins, drugs vaccines/adjuvants, chemotherapeutic agents, cosmetics, and other chemicals. The method serve as preditive platforms for identifying bisignatures for the transition from normal homeostasis to disease development.

### ABBREVIATIONS AND DEFINITIONS

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The abbreviation “RWV” refers to rotating wall vessel. The RWV is an optimized suspension culture method in which cells are grown on extracellular matrix-coated microcarrier beads in cylindrical bioreactors, called slow turning lateral vessels (STLV) or high aspect ratio vessels (HARV), in physiologically relevant low fluid-shear conditions (Nickerson et al., 2004b).

The term “cell lines” means cells that are derived from an organism and maintained in an environment that provides the conditions necessary for division (typically referred to as culturing). “Cell lines” can be maintained indefinitely in a constantly dividing state (i.e., immortalized) or maintained in culture up to cell senescence (e.g., approximately 50 cell divisions). “Primary cells” are cells taken directly from living tissue (e.g., biopsy material) and established for growth in vitro.

“Fluid shear” is shear stress created by a fluid along a solid boundary. In biological systems, a solid boundary can be a tissue, such as an epithelial layer. Biomechanical forces such as fluid shear are known to influence cellular differentiation and development.

As used herein, “low fluid shear environment” means an environment with low fluid shear along with other physical and biological factors that affect the metabolism, signaling, and/or other interactions of a cell. A “low fluid shear environment” includes shear stress rates ranging from about 0 dynes/cm² to about 10.0 dynes/cm.² A “low fluid shear environment” simulates the environment encountered during spaceflight, in certain spaceflight analogs including in a bioreactor, such as a RWV bioreactor, and in certain areas of the body, including mucosal tissues of the gastrointestinal, lung and urogenital tracts, which are primary sites of infection.

As used herein, the term “about” is used to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 10 percent up or down (higher or lower).

As used herein, the term “tissue system” means a dynamic, integrated group of cells in a definite organization that carry out a function. For example, an immune tissue system is a dynamic, integrated group of cells, including, but not limited to, lymphoid cells, innate immune cells, epithelial cells, and effector molecules, which carry out the function of host protection and defense.

Methods of Producing Three-Dimensional Tissue Systems with Physiologically Relevant Characteristics

The methods and systems disclosed herein relate to producing three-dimensional, physiologically relevant tissue systems. In one embodiment, the tissue system is an immunocompetent tissue system. In one embodiment, the methods comprise introducing an immune cell and at least one other cell type, including immune cells, into a low fluid shear environment. Next, the immune cell and the at least one other cell type are co-cultured under conditions selected to produce a three-dimensional immune tissue system with one or more physiologically relevant characteristics.

The term “physiologically relevant characteristics” refer to characteristics of tissue systems that are similar both structurally and functionally to those found in in vivo tissues, including human tissues. The methods produce three-dimensional tissue systems with similar cellular organization, morphology, and histology to in vivo tissue systems. Physiologically relevant characteristics include, but are not limited to, one or more differentiated and functional cells; production of extracellular matrix components; assembly into relevant three-dimensional aggregates; and physiologically relevant cell type ratios. Physiologically relevant characteristics can differ depending on the specific tissue system. In one embodiment, for an immunocompetent tissue system, physiologically relevant characteristics further include differentiated immune cells (for example, macrophages) that conduct immune cell functions, such as phagocytosis and production of inflammatory mediators.

In another embodiment, for an immunocompetent tissue system with epithelial cells, physiologically relevant characteristics can include a differentiated epithelium, one or more functional macrophage-like cells, a localization of macrophage-like cells on (or beneath) the epithelial surface, production of one or more extracellular matrix components, and a physiologically relevant macrophage-to-epithelial cell ratio. The methods produce tissue systems that are structurally and functionally similar to endogenous mucosa.

The low fluid shear environment enables the development of three-dimensional tissue systems, including immunocompetent co-culture systems containing human immune cells, that display physiologically relevant characteristics similar to in vivo human tissues. Low shear environments can be generated in several ways. For example, the optimized suspension culture RWV bioreactor (including the high-aspect rotating vessel (HARV) and slow turning/transfer lateral vessel (STLV)) generates such a low shear environment. In addition, the conditions encountered during spaceflight and in certain spaceflight analogs provide the appropriate low fluid shear environment. Use of one or a combination of these low shear environments in the disclosed methods is contemplated.

Embodiments of the methods include culturing immune cells and at least one other cell lines from particular tissues, including additional immune cells and epithelial cells. Examples of epithelial cells or cell lines include, but are not limited to alveolar, bronchial, small intestinal, large intestinal, cervical, vaginal, urogenital tract, gastrointestinal tract, and respiratory tract epithelial cells from healthy and/or diseased subjects. The term “epithelial” encompasses cells derived from developmental lineages that are epithelial, endothelial, or mesothelial. Examples of immune cells or cell lines include, but are not limited to, monocytes, astrocytes, neuronal cells, macrophages, dendritic cells, B cells, T cells, natural killer cells, basophils, eosinophils, and neutrophils from healthy and/or diseased subjects.

In one embodiment, the methods include culturing the immune cell and/or the at least one other cell type in a monolayer before placing in the low fluid shear environment. In another embodiment, the methods include developing the immune cell and/or at least one other cell type into three-dimensional cells before placing in the low fluid shear environment.

In one embodiment, the methods include first placing the immune cell in the low fluid shear environment, first placing the at least one other cell type in the low fluid shear environment, or placing the immune cell and the at least one other cell type simultaneously in the low fluid shear environment.

In one embodiment, methods of producing a three-dimensional, physiologically relevant immune tissue system includes first culturing epithelial cells in a monolayer. Next, the epithelial cells are placed in a low fluid shear environment for a time period such that the epithelial cells develop into mature three-dimensional epithelial cells. Then, the mature three-dimensional epithelial cells are co-cultured with undifferentiated immune cells in the low fluid shear environment under conditions selected to produce a three-dimensional immune tissue system with one or more physiologically relevant characteristics. In some embodiments, the undifferentiated immune cells (e.g., monocytes) are pre-differentiated into macrophages with chemical treatment prior to placement in the low fluid shear environment. In other embodiments, the undifferentiated immune cells autonomously differentiate into macrophages in the low fluid shear environment. Physiologically relevant characteristics include one or more differentiated cells, production of extracellular matrix components, and physiologically relevant cell type ratios. In certain embodiments, physiologically relevant characteristics include of a differentiated epithelium, one or more functional macrophage-like cells, a localization of macrophage-like cells on (or beneath) the epithelial surface, production of one or more extracellular matrix components, and a physiologically relevant macrophage-to-epithelial cell ratio.

In some embodiments, undifferentiated immune cells (e.g., monocytes) are first cultured in the low fluid shear environment, and epithelial cells are added to the low fluid shear environment. In other embodiments, the epithelial cells are first cultured in the low fluid shear environment, and the undifferentiated immune cells are added to the low fluid shear environment. In other embodiments, the undifferentiated immune cells and epithelial cells are placed simultaneously in the low fluid shear environment.

In other embodiments, immune cells including astrocytes and neuronal cells are co-cultured under conditions selected to produce a three-dimensional immune tissue system with one or more physiologically relevant characteristics. In one embodiment, neuronal cells are placed in the low fluid shear environment, and then astrocytes are placed in the low fluid shear environment. In another embodiment, astrocytes are first placed in the low fluid shear environment, and then neuronal cells are added. In another embodiment, neuronal cells and astrocytes are placed in the low fluid shear environment simultaneously.

In other embodiments, astrocytes, neuronal cells, and monocytes are co-cultured under conditions selected to produce a three-dimensional immune tissue system with one or more physiologically relevant characteristics.

In additional embodiments, the methods comprise placing the epithelial cells into a low fluid shear environment for a time period. The time period allows the cells to develop into a mature three-dimensional group of cells or tissues. The period of time that the cells can be cultured in the low shear environment is from about 24 hours to about 1 year, from about 24 hours to about 10 months, from about 24 hours to about 6 months, from about 24 hours to about 2 months, from about 24 hours to about 1 month, from about 24 hours to about 1 week, from about 24 hours to about 120 hours, from about 36 hours to about 108 hours, from about 48 hours to about 96 hours, from about 60 hours to about 84 hours, and is about 72 hours. In certain embodiments, the period of time is about 5 days to about 25 days. In other embodiments, the period of time is about 9 days to about 20 days. In particular embodiments, the period of time is about 9 days.

Embodiments of the methods disclosed herein also include co-culturing three-dimensional epithelial cells with undifferentiated immune cells in the low fluid shear environment, and the undifferentiated immune cells then undergo spontaneous differentiation to become immunocompetent. The immune cells can be undifferentiated monocytes, macrophages (i.e., differentiated monocytes), dendritic cells (i.e., differentiated monocytes), astrocytes, neuronal cells, neutrophils, eosinophils, basophils, and lymphocytes, including T lymphocytes, B lymphocytes, and natural killer cells from healthy and/or diseased subjects. In other aspects, the immune cells are differentiated using well-known chemical treatments prior to incorporating into the three-dimensional tissue system. In other embodiments, the methods include co-culturing three-dimensional epithelial cells with differentiated immune cells in the low fluid shear environment.

In certain embodiments, the cells are immune cells, epithelial cells, and other cell types, such as stem cells. In some embodiments, the cells are derived from primary cells from healthy mammals (e.g., humans and non-human primates). In particular aspects, the epithelial cells and the immune cells are derived from human cell lines. For example, epithelial cell lines include, but are not limited to, A549, Int-407 (small intestinal epithelial cells), HT-29, V19I, 5637, HT29 (colonic cells) and HCT-8. Immune cell lines include, but are not limited to, U937 (monocytes), THP-1 (monocytes), neuronal cells such as SH-SY5Y (neuroblastoma), and astrocytes (HTB-14/U87-MG astrocytoma-glioblastoma).

In certain aspects, the methods further include co-culturing the three-dimensional epithelial cells and the undifferentiated immune cells in the low fluid shear environment under conditions selected to produce a three-dimensional immune tissue system with physiologically relevant characteristics. The conditions include, but are not limited to, the culture medium, temperature, pH, the fluid shear, oxygen levels, composition of the extracellular matrix, and time in the low fluid shear environment. In some embodiments, the culture medium is GTSF-2 with and without antibiotics, MEM, DMEM, EME, and RPMI (all with or without antibiotics). In another embodiment, the culture medium is with or without Fungizone. In another embodiment, the culture medium is with or without serum. Extracellular matrix components are collagen-based biodegradable matrices that include, but are not limited to, collagens, glycoproteins laminin, fibronectin, tenascin, elastin, a number of proteoglycans and glycosaminoglycans. Extracellular matrix material, such as submucosal tissue, can be obtained from various sources known in the art. Commercially available ECM products, such as MaxGel Human ECM, may be used in the disclosed methods.

In another embodiment, the method further includes conducting biochemical analyses on the three-dimensional immune tissue system to determine that the tissue system has one or more physiologically relevant characteristics. These biochemical analyses are well-known in the art and include, but are not limited to, microscopy, flow cytometry analysis, cell viability assay, phagocytosis assay, inflammatory response profiling of cytokines/chemokines, Toll-like receptors, NODs, etc, antibody production (sIgA, IgG), Th1 and Th2 type responses, T helper and cytotoxic T cell responses, biosignature profiling at the transcriptomic, proteomic and metabolomic levels.

In another embodiment, the time period for introducing the monolayer epithelial cells into an environment with low fluid shear conditions to develop into mature three-dimensional epithelial cells ranges from about 1 day to about 40 days, from about 2 days to about 29 days, from about 5 days to about 25 days, from about 9 days to about 20 days, or about 9 days.

In another embodiment, the ratio of the undifferentiated immune cells and the mature three-dimensional epithelial cells can range broadly from about 1:1×10⁹ to about 1×10⁹:1, 1:1×10⁸ to about 1×10⁸, 1:1×10⁷ to about 1×10⁷, 11:1×10⁶ to about 1×10⁶:1, 1:1×10⁵ to about 1×10⁵, 1:1×10⁴ to about 1×10⁴, 1:1×10³ to about 1×10³, 1:1×10² to about 1×10², 1:10 to about 10:1, 1:5 to about 5:1, 1:4 to about 4:1, 1:3 to about 3:1, from about 1:2 to about 2:1, or is about 1:1. Similar ratio ranges are contemplated for other cell types, including immune cells, epithelial cells, stem cells, etc.

In another embodiment, the low fluid shear environment is provided by a bioreactor. Suitable ranges for the low fluid shear environment ranges from about 0 dynes/cm² to about 10.0 dynes/cm², from about 0.1 dynes/cm² to about 1.9 dynes/cm², from about 0.2 dynes/cm² to about 1.8 dynes/cm², from about 0.3 dynes/cm² to about 1.7 dynes/cm², from about 0.4 dynes/cm² to about 1.6 dynes/cm², from about 0.5 dynes/cm² to about 1.5 dynes/cm², from about 0.6 dynes/cm² to about 1.4 dynes/cm², and from about 0.7 dynes/cm² to about 1.3 dynes/cm², from about 0.8 dynes/cm² to about 1.2 dynes/cm², and from about 0.9 dynes/cm² to about 1.1 dynes/cm.²

In one embodiment, the bioreactor can be a RWV bioreactor. In one embodiment, the RWV bioreactor can be a STLV. In another embodiment, the RWV bioreactor can be a HARV.

In one embodiment, the rotation speed of the bioreactor ranges from about 10 rotations per minute (rpm) to about 30 rpm, from about 15 rpm to about 25 rpm, and is about 20 rpm.

In one embodiment, the time for co-culturing different cell types (e.g., epithelial cells with monocytes) in the bioreactor ranges from hours to years. In one embodiment, the time ranges from about 24 hours to about 1 year, from about 24 hours to about 10 months, from about 24 hours to about 6 months, from about 24 hours to about 2 months, from about 24 hours to about 1 month, from about 24 hours to about 1 week, from about 24 hours to about 120 hours, from about 36 hours to about 108 hours, from about 48 hours to about 96 hours, from about 60 hours to about 84 hours, and is about 72 hours.

In another embodiment, the culture medium is GTSF-2 with and without antibiotics, MEM, DMEM, EME, and RPMI (all with or without antibiotics). In another embodiment, the culture medium is with or without Fungizone.

In another embodiment, the one or more physiologically relevant characteristics are selected from the group consisting of a highly differentiated epithelium, one or more functional macrophage-like cells, a localization of macrophage-like cells on (or beneath) the epithelial surface, and a physiologically relevant macrophage-to-epithelial ratio.

In another embodiment, the one or more functional macrophage-like cells were autonomously differentiated.

In another aspect, the physiologically relevant macrophage-to-epithelial cell ratio ranges from about 1:30 to about 1:40, from about 1:32 to about 1:38, from about 1:33 to about 1:37, and is about 1:32.

In one aspect, the tissue system is comprised of both lung epithelial cells and immune cells co-cultured in the low fluid shear environment. The lung epithelial-immune cell tissue equivalent is characterized as having cellular organization, morphology, and histology similar to in vivo lung epithelial tissue. For example, the tissue system includes immunocompetent cells in the epithelial tissue, which results in immunocompetence of the tissue system. In another aspect, the tissue system is comprised of both intestinal epithelial cells and immune cells co-cultured in the low fluid shear environment. The intestinal epithelial-immune cell tissue equivalent is characterized as having cellular organization, morphology, and histology similar to in vivo intestinal tissue. In another aspect, the tissue system is comprised of astrocytes, neuronal cells, and/or monocytes that develop into a three-dimensional immune tissue system with one or more physiologically relevant characteristics.

Embodiments of the methods disclosed herein improve the predictive capability of tissue culture systems to mimic human and patient-specific responses, decreasing expensive animal use (through reduction, replacement and refinement) and human clinical testing. By serving as better predictors of human and patient-specific responses, these three-dimensional tissue systems allow personalized medicine to better predict in vivo human responses to, for example, infectious pathogens, toxins, drugs/therapeutics, vaccines/adjuvants, chemotherapeutic agents, cosmetics and other chemicals and identify biomarkers for transition from normal homeostasis to disease.

### Three-Dimensional Cell Culture

Through more optimal cell-matrix and cell-cell interactions, three-dimensional models of various tissues display significant morphological, phenotypic and molecular aspects of the parental tissue. These characteristics include, but are not limited to, establishment of apical and basolateral polarity, enhanced expression of tight junctions, extracellular matrix and brushborder proteins, highly localized mucin production, and pluripotent properties. Since cellular differentiation and tissue architecture is more in vivo-like in three-dimensional models as compared to conventional two-dimensional monolayers, three-dimensional tissue systems have the potential to mimic and predict cellular responses following exposure to foreign agents such as pathogens, candidate drugs and toxins (Mueller-Klieser, 1997, Abbott, 2003, Schmeichel et al., 2003, Nickerson et al., 2004a).

In one aspect, the methods disclosed include co-culturing immune cells and at least one other cell type, including additional immune cells, into three-dimensional, functional cells. In one embodiment, the cells are developed into three-dimensional cells by using a scaffold or support surface. Scaffolds may be made of any suitable material, such as glass, plastic, foam, fiber meshes or bioscaffolds, including decelularized bioscaffolds. Other suitable support surfaces include tubes, sutures, membranes, films, microparticles, and microcarrier beads. The microcarriers may include, but are not be limited to, Cytodex 1, Cytodex 3, Microhex, Cultisphere, Solohill collagen, Solohill FACT, Solohill hillex II, Solohill pronect or Solohill plastic.

In one embodiment, the scaffold is coated with one or more extracellular matrix components. Extracellular matrix components are collagen-based biodegradable matrices that include, but are not limited to, collagens, glycoproteins laminin, fibronectin, tenascin, elastin, a number of proteoglycans and glycosaminoglycans. Extracellular matrix material, such as submucosal tissue, can be obtained from various sources known in the art. For example, intestinal tissue can be harvested from animals raised for meat production, including pigs, cattle and sheep or other warm-blooded vertebrates. Commercially available ECM products, such as MaxGel Human ECM, may be used in the disclosed methods.

### RWV Bioreactor

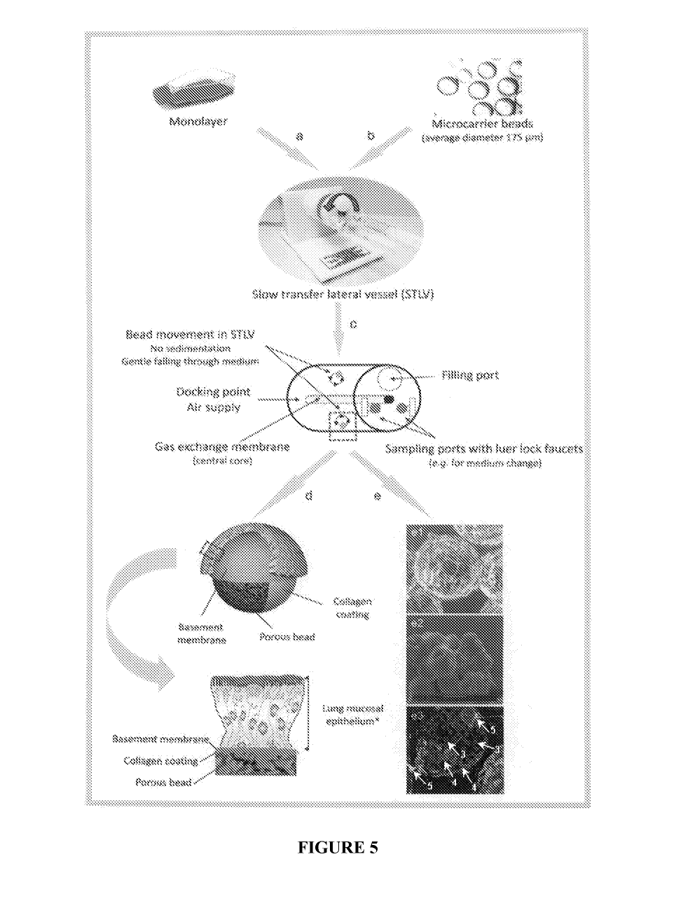

The RWV is an optimized suspension culture method in which cells are grown on extracellular matrix-coated microcarrier beads in cylindrical bioreactors, called slow turning lateral vessels (STLV) or high aspect ratio vessels (HARV), in physiologically relevant low fluid-shear conditions (Nickerson et al., 2004b) (FIG. 5). In FIG. 5, during step (a), confluent monolayers are trypsinized and introduced into a slow transfer lateral vessel (STLV). In step (b), extracellular matrix-coated porous microcarrier beads are introduced into the STLV with the confluent layers. In step (c), the STLV is rotated along a horizontal axis, which offsets sedimentation. This rotation results in gentle falling of the beads and cells through the culture medium within a restricted orbit. The RWV creates a physiological low fluid-shear environment that allows the cells to grow in three-dimensions, aggregate based on natural cellular affinities, and differentiate into human surrogate tissue-like models. A central core gas permeable membrane provides adequate gas exchange. As shown in the enlarged representation step (d), epithelial cells form a complex tissue system on a microcarrier bead using the RWV. Representation step (e) shows three-dimensional aggregates of the lung epithelium taken out of the STLV and analyzed with, e.g., light microscopy (e1), electron microscopy (e2), and confocal laser scanning microscopy (CLSM (e3)). In the latter image, cell nuclei are stained blue, shown as 3 (DAPI), the cytoskeleton red, shown as 4 and mucus production (MUC5AC) yellow, shown as 5. In some embodiments, the STLV provides the low fluid shear environment. In other embodiments, the HARV provides the low fluid shear environment. In other embodiments, the cells are co-cultured in low fluid shear environments provided by a combination of HARV, STLV, other bioreactors, and/or a spaceflight environment.

The HARV and the STLV differ in their aeration source. The low sedimental physiological fluid shear conditions are relevant to those encountered in vivo and during spaceflight culture, and the conditions mimic the major characteristics that are operative in the human host and drive differentiated form and functionality of human tissues, e.g., three-dimensional architecture, multi-cellular complexity, cell shape and biomechanical forces. RWV-derived three-dimensional immunocompetent co-culture models allow the contribution of every different cell type in a tissue to be studied in the disease response to pathogen and toxin challenge (biological or chemical) or drug, vaccine/adjuvant, therapeutic, cosmetic or other treatments—and thus serve as predictive human surrogate platforms to understand the transition from normal to disease development.

RWV-derived three-dimensional tissue models have been developed, some of which have been used for infectious disease studies. Examples include the human lung (Carterson et al., 2005, Crabbe et al, 2011), small intestine (Nickerson et al., 2001; Straub et al, 2007), colon (Carvalho et al., 2005, Honer zu Bentrup et al., 2006; Radtke et al, 2010), liver (Sainz et al., 2009) and bladder (Smith et al., 2006). The present disclosure uses the bioreactor environment to co-culture an immune cell line and at least one other cell line to produce three-dimensional, physiologically relevant tissue systems. In one embodiment, the three-dimensional, physiologically relevant tissue system is immunocompetent.

The design of the RWV bioreactor (FIG. 5) is based on the principle that organs and tissues function in a three-dimensional environment. This optimized form of suspension culture is used for growing three-dimensional cell cultures that maintain many specialized features of in vivo tissues (Unsworth et al. Nature Med. 4, 901-907 (1998); Schwarz et al., J. Tissue Cult. Methods 14, 51-57 (1992)). Moreover, the RWV provides a low-fluid-shear growth environment similar to that encountered by pathogens in certain regions of the body (including between the brush border microvilli of epithelial cells and in utero).

The RWV is a cylindrical, rotating bioreactor that is filled with culture medium. The sedimentation of cells in the vessel is offset by the rotating fluid, creating a constant, gentle fall of cells through the medium under conditions of physiologically relevant fluid shear (Nickerson, C., et al. (2004) Microbiol. Mol. Biol. Rev. 68, 345-361).

Fluid shear is a biomechanical force known to influence cellular differentiation and development in mammals. The dynamic culture conditions in the RWV allow cells to grow in three dimensions, to aggregate based on natural cellular affinities (facilitating co-culture of multiple cell types) and to differentiate into three-dimensional tissue-like systems (Nickerson, C. A. & Ott, et al. (2004) ASM News 70, 169-175; Unsworth, B. R. & Lelkes, et al. (1998) Nature Med. 4, 901-907). The RWV design also allows easy manipulation of culture conditions, including, for example, the addition or removal of cells and media at various time points.

Three-Dimensional Co-Culture in the RWV

In one aspect, to initiate three-dimensional cell culture in the RWV, cells can be first grown as conventional monolayers in standard tissue culture flasks (FIG. 5). At the appropriate density, the cells are removed from the flask, re-suspended in medium and incubated in the RWV with porous ECM-coated microcarrier beads (or other scaffolding) for attachment. This allows the cells to respond to chemical and molecular gradients in three dimensions, in a manner akin to the response of the tissue in vivo. The cell-bead complexes are then introduced to the RWV and rotation is initiated. Other types of scaffolding known in the art can be used in the RWV to support cellular aggregation and differentiation. For example, a recent report used novel hyaluronan hydrogel-coated microcarriers as scaffolds for RWV-derived 3D intestinal models, which allowed for enzyme-free cell detachment, with applications in tissue regeneration and transplantation (Skardal et al., Biomaterials 31, 8426-8435 (2010)). Bioscaffolds consisting of submucosa have also been used (Caravalho et al, 2005). Moreover, recellularization of decellularized bioscaffolds can be used. In addition, cells can also spontaneously aggregate under scaffold-free conditions during RWV culture. One of ordinary skill in the art can choose the particular scaffold material and whether to use scaffolds at all.

Various cell culture media can be used in the disclosed methods, including, but not limited to, GTSF-2 with and without antibiotics (and with or without fungizone, serum, ITS, short chain fatty acids, etc.), MEM, DMEM, EMEM, RPMI, AIMS, IMDM (all with or without antibiotics). Both chemically defined media and media that is serum-supplemented can be used with the disclosed methods. In some embodiments, a combination of different types of media or one specific media, such as GTSF-2 without antibiotics or fungizone, can be used.

### Exposing RWV-Derived Three-Dimensional Cell Co-Cultures to Infectious Agents and Compounds of Interest

In one aspect, a method of assessing disease pathogenesis in a three-dimensional, physiologically relevant immune tissue system is provided.

The method comprises introducing an infectious agent, such as a pathogen, or a compound into a three-dimensional, physiologically relevant tissue system. The three-dimensional, physiologically relevant immune system is produced under low fluid shear conditions and has one or more physiologically relevant characteristics. Next, the disease effects of the pathogen or compound on the three-dimensional, physiologically relevant immune tissue system are tested using methods known in the art. The compound includes, but is not limited to, toxins, antimicrobial drugs, adjuvants, and vaccines.

In one embodiment, after the three-dimensional, physiologically relevant immune tissue system described herein are established, the tissue system is exposed to infectious agents and compounds. In one embodiment, this exposure can be accomplished by removing the three-dimensional cells from the low fluid shear environment and distributing them evenly in multi-well plates or other convenient formats for testing. Cells removed from the low fluid shear environment retain their differentiated state long enough to be amenable to a wide range of experimental manipulations, as determined empirically for each cell type by immunohistochemical, histological and functionality assessments. In another embodiment, a pathogen or compound is introduced into the low fluid shear environment simultaneously with the three-dimensional, physiologically relevant immune tissue system.

The disclosed three-dimensional, physiologically relevant immune tissue systems can therefore be incorporated into existing assays used for infection studies, such as assays testing microbial adherence, invasion and intracellular survival; microscopic examination; transcriptomic, proteomic and metabolomic analyses; expression profiling for cytokines and other inflammatory mediators; and flow cytometry (Nickerson, C. A., et al. (2007) J. Neuroimmune Pharmacol. 2, 26-31). For studies that require homogeneous cell suspensions, such as flow cytometry and cell viability assays (Carterson, A. J. et al. (2005) Pseudomonas aeruginosa pathogenesis. Infect. Immun. 73, 1129-1140; Nickerson, C. A. et al. (2001) Infect. Immun. 69, 7106-7120), three-dimensional cell cultures can be removed from the microcarrier beads using conventional enzymatic and non-enzymatic treatments (for example, trypsin or EDTA, respectively).

Although the treatments for flow cytometry analysis can disrupt the delicate three-dimensional architecture, the expression of cellular markers such as those for differentiation and apoptosis can still be quantified at the single-cell level.

In the context of vaccines and adjuvants, for example, embodiments are used to predict the efficacy of a vaccine/adjuvant and its clinical correlates of protection by means of an in vitro challenge with disease agents. In other embodiments, the tissue systems are prepared using human cell lines or primary cells from healthy (i.e., not diseased, uninfected, naive) individuals or from individuals suffering from diseases or infections. “Diseased cells” include virally infected cells, bacterially infected cells, tumor cells, cells from patients with genetics disorders, and cells and tissues affected by a pathogen or involved in an immune-mediated disease, such as, e.g., autoimmune disease.

Embodiments can be used to assess the interaction of substances with the immune system, and thus can be used to accelerate and/or improve the accuracy, predictability, safety and efficacy, for example, of vaccines, adjuvants, drugs, biologics, immunotherapy, cosmetic and chemical development.

### Assessing Host-Pathogen Interactions and Host Responses to Infection

In another aspect, a method of assessing a disease treatment in a three-dimensional, physiologically relevant tissue system is disclosed. The method comprises establishing a disease state in a three-dimensional, physiologically relevant tissue system, which was produced under fluid shear conditions and has one or more physiologically relevant characteristics. Next, a disease treatment is introduced into the three-dimensional, physiologically relevant tissue system. Then, the effect of the disease treatment on the disease is tested using methods known in the art.

Mechanisms of Microbial Pathogenesis.

There are many pathogens that lack practical and representative cell culture or animal models which accurately reflect the host response to infection, and the RWV has enabled researchers to study such pathogens (Alcantara Warren, C. et al. (2008) J. Infect. Dis. 198, 143-149; Duray, P. H. et al. (2005) Invasion of human tissue ex vivo by Borrelia burgdorferi. J. Infect. Dis. 191, 1747-1754; Long, J. P., et al. (1999) In vitro Cell Dev. Biol. Anim. 35, 49-54; Margolis, L. B. et al. (1997) AIDS Res. Hum. Retroviruses 13, 1411-1420; Straub, T. M. et al. (2007) Emerg. Infect. Dis. 13, 396-403). In this regard, three-dimensional models of the intestine have been shown to have in vivo-like expression levels and distribution patterns of key biological surface markers that are directly accessible to pathogens (Nickerson, C. A. et al. (2001) Infect. Immun. 69, 7106-7120; Carvalho, H. M., et al. (2005) Cell. Microbiol. 7, 1771-1781; Honer zu Bentrup, K. et al. (2006) Microbes Infect. 8, 1813-1825; Radtke et. al, 2010, Analysis of interactions of Salmonella type three secretion mutants with 3-D intestinal epithelial cells. PLoS ONE, 2010 Dec. 29; 5(12):e15750) (Table 1), and this contributes to the ability of these models to support productive pathogen infection and replication.

The complexity and in vivo-like characteristics of the disclosed three-dimensional tissue systems make them not only useful systems to investigate the mechanisms involved in, for example, microbial pathogenesis, but also valuable tools to study the host response to, for example, microbial infection. The intestinal and lung mucosa are two major portals of entry for microorganisms. Both tissues have been modelled using the RWV and subsequently used to study enteric and respiratory pathogens, respectively (Carterson, A. J. et al. (2005) Infect. Immun. 73, 1129-1140; Nickerson, C. A. et al. (2001) Infect. Immun. 69, 7106-7120; Honer zu Bentrup, K. et al. (2006) Microbes Infect. 8, 1813-1825; Vertrees, R. A. et al. (2008) Cancer Biol. Ther. 7, 404-412; Straub et al, 2007, Emerg. Infect. Dis. 13, 396-403; Radtke et al, 2010, PLoS ONE, 2010 Dec. 29; 5(12):e15750). The previous models do not use co-culture methods with an immune cell and at least one other cell type to produce three-dimensional immune tissue systems with one or more physiologically relevant characteristics.

A protective and regulated inflammatory response is also crucial for host defense and survival during infection (Imler, J. L. & Hoffmann, et al. (2001) Trends Cell Biol. 11, 304-311; Kabelitz, D. & Medzhitov, et al. (2007) Curr. Opin. Immunol. 19, 1-3; Akira, S., et al. (2006) Cell 124, 783-801; Medzhitov, R. & Janeway, et al. (1997) Curr. Opin. Immunol. 9, 4-9).

The disclosed three-dimensional tissue system is a meaningful predictor of the outcomes of and host responses to in vivo infections.

### Kits

The disclosure relates to a kit for producing a three-dimensional, physiologically relevant tissue system. In one aspect, the kit comprises immune cell line and at least one other cell line; and informational material for producing a three-dimensional, physiologically relevant tissue system.

In one embodiment, the kit comprise an epithelial cell line; monocytes; and informational material for producing a three-dimensional, physiologically relevant tissue system. In other embodiments, the kit comprise astrocytes, neuronal cells, and informational material for producing a three-dimensional, physiologically relevant tissue system. Examples of intestinal cell lines include, but are not limited to alveolar, bronchial, small intestinal, large intestinal, cervical, and vaginal epithelial cells. Immune cell lines include, but are not limited to, undifferentiated monocytes, macrophages (i.e., differentiated monocytes), dendritic cells (i.e., differentiated monocytes), astrocytes, neuronal cells, neutrophils, eosinophils, basophils, and lymphocytes, including T lymphocytes, B lymphocytes, and natural killer cells from healthy and/or diseased subjects.

In other embodiments, the kit further comprises monocytes. The informational material can be descriptive, instructional, marketing or other material that relates to the methods described herein.

The informational material of the kits is not limited in its form. In some instances, the informational material can include information about how to produce a three-dimensional, physiologically relevant tissue system, including conditions of the bioreactor appropriate for differentiation of the cells and one or more biochemical analyses to determine that the three-dimensional tissue has one or more physiologically relevant characteristics.

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about the nanoparticles therein and/or their use in the methods described herein. The informational material can also be provided in any combination of formats.

In addition to the cell lines (for example, an epithelial cell line and monocytes), the kit can include other ingredients, including but not limited to cell culture medium, extracellular matrix components, and scaffolds. Extracellular matrix components are collagen-based biodegradable matrices that include, but are not limited to, collagens, glycoproteins laminin, fibronectin, tenascin, elastin, a number of proteoglycans and glycosaminoglycans. Extracellular matrix material, such as submucosal tissue, can be obtained from various sources known in the art. Commercially available ECM products, such as MaxGel Human ECM, may be used in the disclosed methods. Scaffolds may be made of any suitable material, such as glass, plastic, foam, fiber meshes or bioscaffolds, including decellularized bioscaffolds. Other suitable support surfaces include tubes, sutures, membranes, films, microparticles, and microcarrier beads. The microcarriers may include, but are not be limited to, Cytodex 1, Cytodex 3, Microhex, Cultisphere, Solohill collagen, Solohill FACT, Solohill hillex II, Solohill pronect or Solohill plastic.

### EXAMPLES

### Example 1

### Three-Dimensional A549-U937 Co-Culture

The study described in this Example developed a lung tissue model that reflected the parental tissue with regard to three-dimensional architecture, differentiation and multi-cellular complexity to incorporate the role of alveolar epithelium and macrophages, and their interactions, in the overall response of each cell type to bacterial virulence factors, such as QS signals.

A RWV-derived three-dimensional co-culture model of epithelial cells and immune cells was developed. The previously developed three-dimensional monotypic alveolar A549 lung epithelium model (Carterson et al., 2005) only included one cell type. In this disclosure, the methods produce three-dimensional tissue systems with physiologically relevant characteristics; this system is a multi-cellular immunocompetent model that includes differentiated immune cells, such as macrophages. Macrophages are an important innate immune defense cell of the lung. The expression of specific cell surface markers and phagocytic activity demonstrated the presence of naturally differentiated macrophages in the co-culture model.

This three-dimensional co-culture model was applied to study the cytotoxic effects of 3-oxo-C₁₂ HSL on macrophages and epithelial cells in a more in vivo-like tissue model, showing surprising discrepancies with previously reported in vitro studies using single cell types. The multi-cellular complexity and biologically relevant cellular organization of this three-dimensional co-culture model, which integrated the pivotal role of mononuclear phagocytic cells in the antibacterial defense and mediation of inflammation, more adequately mimicked the in vivo lung response to respiratory pathogens and their virulence factors.

Quorum sensing (QS) signals mediate inter-cellular communication among bacteria, which coordinates population gene expression and triggers the production of virulence factors. The opportunistic pathogen Pseudomonas aeruginosa, responsible for terminal lung infections in patients with cystic fibrosis, predominantly produces the QS molecules N-(3-oxododecanoyl)-Lhomoserine lactone (3-oxo-C₁₂ HSL) and N-butyryl-L-homoserine lactone (Williams et al., 2009, Winstanley et al., 2009). The quorum sensing signal 3-oxo-C₁₂-HSL), produced by Pseudomonas aeruginosa, exerts cytotoxic effects in macrophages in vitro, which is believed to affect host innate immunity in vivo.

QS regulates P. aeruginosa virulence determinants, such as biofilm formation and the production of pigments, proteases and exotoxins, all contribute to lung pathology (Williams et al., 2009, Winstanley et al., 2009). In addition to inter-species gene regulation, 3-oxo-C₁₂ HSL acts across kingdoms, as it was shown to exert immunomodulatory and cytotoxic effects in eukaryotes (Telford et al., 1998, Tateda et al., 2003, Williams et al., 2004, Kaufmann et al., 2008).

A significant loss in viability of pure cultures of monocytes and macrophages was demonstrated, when exposed to high concentrations of 3-oxo-C₁₂ HSL, while a variety of epithelial cell lines, including cultures of alveolar epithelial cells, were not affected (Tateda et al., 2003). Indeed, pure cultures of airway epithelial cells were found to produce paraoxanase enzymes that inactivate 3-oxo-C₁₂ HSL through a lactonase mechanism (Ozer et al., 2005). However, these past studies were performed on single cell type cultures of either monocytes/macrophages or epithelial cells, respectively, which do not reflect the multi-cellular complexity of the parental tissue in vivo.

This becomes of particular importance given the fact that mutual interactions and cytokine networking between alveolar epithelium and macrophages are essential for the innate defense of the lung to bacterial infection (Standiford et al., 1991, Krakauer, 2002, Amano et al., 2004, Kannan et al., 2009). Besides the role of alveolar macrophages in the elimination of the daily-inhaled bacterial load, secretion products from macrophages trigger immune responses in alveolar epithelial cells, such as cytokine production (Standiford et al., 1991, Krakauer, 2002).

On the other hand, alveolar epithelial cells increase the immune function of alveolar macrophages in response to infection with P. aeruginosa and other opportunistic pathogens, which is partly mediated through the intercellular adhesion molecule ICAM-1 (Amano et al., 2004, Kannan et al., 2009). The expression of ICAM-1 on the apical surface of alveolar epithelial cells induces phagocytic capabilities of alveolar macrophages and facilitates their migration along the epithelial cell surface (Paine et al., 2002). P. aeruginosa virulence factors, such as lipopolysaccharides, lipopeptides, porins and phenazins, up-regulate ICAM-1 expression in different host cell types, including airway epithelium (Perfetto et al., 2003, Greene et al., 2005).

The three-dimensional co-culture tissue system of alveolar epithelium and macrophages using the rotating wall vessel (RWV) bioreactor in this Example was developed by adding undifferentiated monocytes to RWV-derived alveolar epithelium. This three-dimensional tissue system expressed important architectural/phenotypic hallmarks of the parental tissue, as evidenced by highly differentiated epithelium, spontaneous differentiation of monocytes to functional macrophage-like cells, localization of these cells on the alveolar surface, and a macrophage-to-epithelial cell ratio relevant to the in vivo situation. Co-cultivation of macrophages with alveolar epithelium counteracted 3-oxo-C₁₂-HSL-induced cytotoxicity via removal of quorum sensing molecules by alveolar cells. Furthermore, 3-oxo-C₁₂-HSL-exposed macrophages should be able to mediate innate immunity since 3-oxo-C₁₂-HSL induced the intercellular adhesion molecule ICAM-1 in both alveolar epithelium and macrophages. This Example demonstrates the importance of multi-cellular organotypic models to integrate the role of different cell types in overall lung homeostasis and disease development in response to external factors.

### In Vivo-Like Characteristics of the Developed Three-Dimensional A549-U937 Co-Culture

Expression of Epithelial Differentiation Markers.

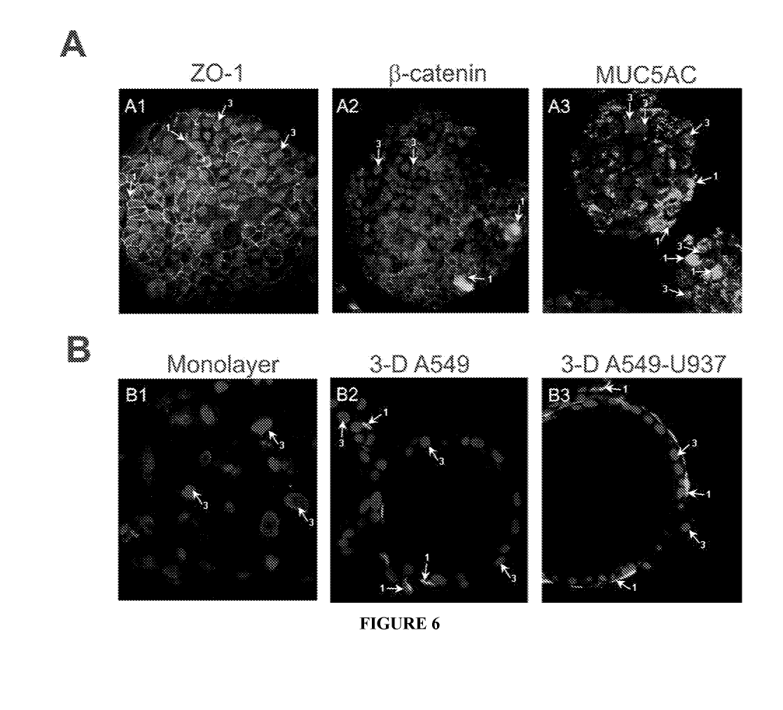

Three-dimensional A549-U937 co-culture aggregates were stained with selected markers for the assessment of epithelial cell differentiation. In FIG. 6A, the three-dimensional A549-U937 model (72 hour co-cultivation) is stained with antibodies against ZO-1 (A1), β-catenin (A2) and MUC5AC (A3). The respective antigens are stained orange ( ), while blue staining (shown as 3) represents the cell nuclei (DAPI). Images are based on 400× magnifications. Tight junctional markers ZO-1 and β-catenin were expressed in the three-dimensional A549-U937 co-culture aggregates at contact zones between adjacent cells, indicative of differentiated cells (FIG. 6A1-A2). Furthermore, three-dimensional A549-U937 co-cultures displayed a well-localized MUC5AC (secreted mucin) staining covering a majority of the apical surface (FIG. 6A3). FIGS. B1-3 show ICAM-1 staining of A549 confluent monolayers (B1), three-dimensional A549 monotypic cultures (B2), and three-dimensional A549-U937 co-cultures. Three-dimensional A549 monotypic cultures showed occasional apical ICAM-1 staining, while ICAM-1 was highly and uniformly expressed mainly at the apical cell side of the alveolar epithelium co-cultured with U937 cells (FIG. 6B2-B3). This increased ICAM-1 expression was confirmed by flow cytometry analysis with a 2.1±0.7-fold increase on the three-dimensional A549 cells after 72 hours of co-cultivation with U937 cells when compared to three-dimensional monotypic A549 cells. In contrast, when A549 cells were grown as monolayers, ICAM-1 staining was weak and localized in the cytoplasm of some cells (FIG. 6B1). Since ICAM-1 expression at the apical side of alveolar epithelial cells is correlated with their differentiation status (Christensen et al., 1993, Kang et al., 1993), the latter results indicated a higher cellular differentiation of three-dimensional A549 alveolar epithelial cells when co-cultured with mononuclear phagocytes as compared to monotypic three-dimensional A549 cells or conventional monolayers.

Architectural Organization of Three-Dimensional Co-Cultures.

The three-dimensional alveolar epithelial cells were present as a unilayer on the surface of the microcarrier beads, which is physiologically relevant to the parental tissue (FIG. 6 B2-B3). For detection of U937 cells in the three-dimensional co-culture, the well-defined leukocyte plasma membrane phosphatase CD45 marker was used, which is expressed in all mononuclear phagocytic cells (Trowbridge et al., 1994) but not in epithelial cells.

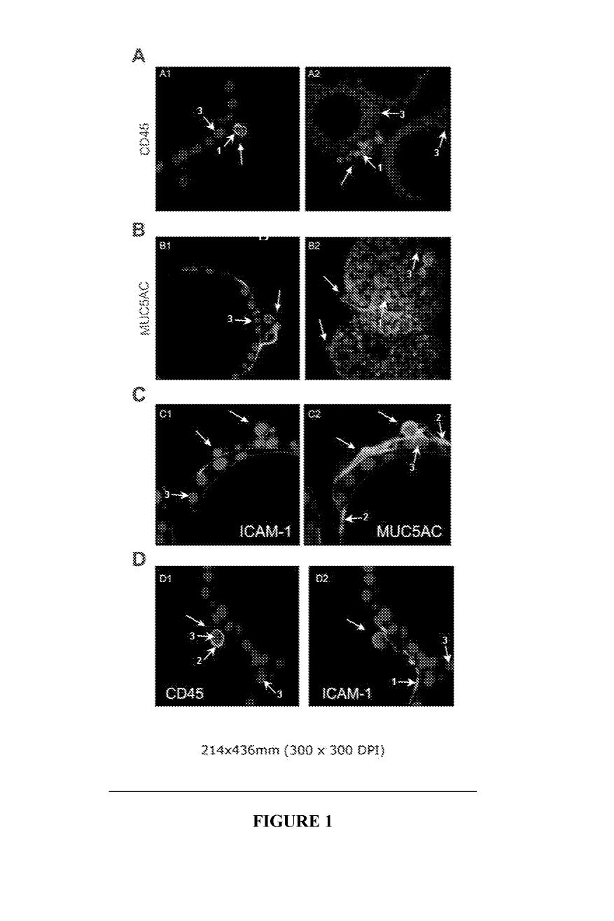

After 72 hour co-cultivation of three-dimensional A549 cells with U937 monocytes, CD45 positive cells lining the surface of the alveolar epithelium were detected with confocal laser scanning microscopy (CLSM) (FIG. 1A), which was relevant to their location within the alveolar surfactant film in vivo (Lohmann-Matthes et al., 1994). FIG. 1A represents immunofluorescence staining of the three-dimensional A549-U937 for CD45 (orange, shown as 1). Cell nuclei are stained blue, shown as 3 (DAPI) and images are based on 400× magnifications. White arrows point out macrophage-like cells. In FIG. 1A, while CD45-positive cells were mostly found as single cells adhering to the epithelial surface (FIG. 1A1), groups of cells were occasionally visualized (FIG. 1A2). To understand the nature of monocyte adherence to the alveolar epithelium, co-cultures were stained for MUC5AC. MUC5AC staining (orange, shown as 1) of the co-culture revealed that adherent cells were completely embedded in the mucus layer of the epithelial cells (FIG. 1B), which is again relevant to the in vivo condition. Dual labeling of the co-cultures with ICAM 1 (orange, shown as 1)—MUC5AC (green, shown as 2) (FIG. 1C) and ICAM 1—CD45 (green, shown as 2) (FIG. 1D) confirmed that mucus-embedded cells were monocytes/macrophages and not epithelial cells. Notably, ICAM-1 expression on monocytes and macrophages is localized throughout the entire circumference of the cell membrane, which can clearly be distinguished from its apical staining in differentiated alveolar epithelium (FIG. 1D2-C1). FIGS. A1, B1, C1, C2, D1 and D2 are single plane images, while FIGS. A2 and B2 are assemblies of Z-stacks.

ICAM-1 Expression and Phagocytic Activity of Adherent U937 Cells.

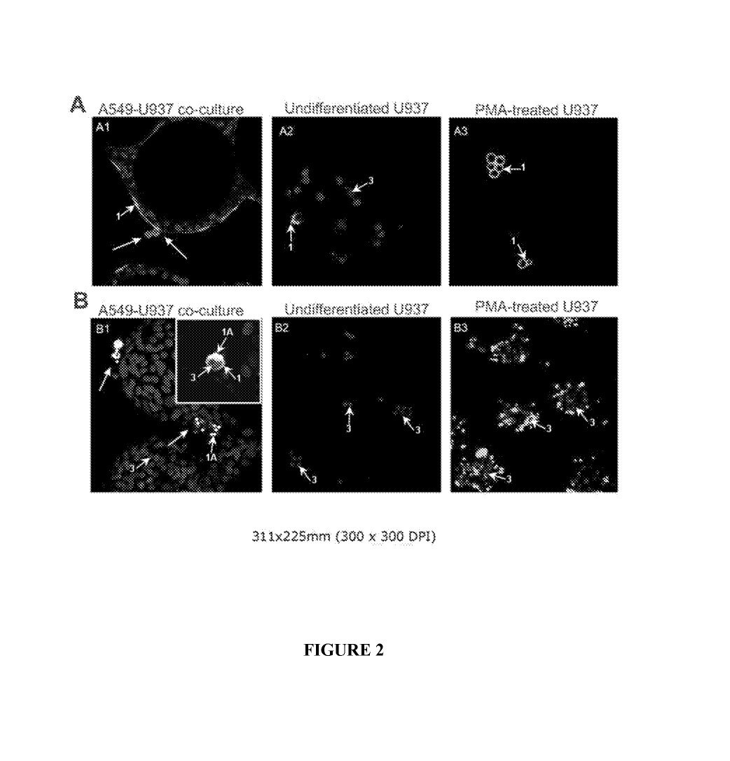

ICAM-1 was chosen as a marker to assess natural differentiation of monocytes to macrophages in the three-dimensional coculture model. Indeed, when monocytes were cultured alone, a majority stained weakly positive for ICAM-1, while macrophages differentiated with phorbol 12-myristate 13-acetate (PMA) showed strong ICAM-1 labeling (FIG. 2A2-3). FIGS. 2A1-3 show ICAM-1 expression on co-cultured alveolar epithelium and monocytes/macrophages in the three-dimensional A549-U937 co-culture (A1) compared to undifferentiated U937 monocytes (A2) and PMA-differentiated macrophages (A3). White arrows point out macrophage-like cells. Cell nuclei are stained blue, shown as 3 (DAPI), and the images are based on 400× magnifications. Imaging of three-dimensional A549 aggregates co-cultured with monocytes for 72 hours indicated that cells adhering to the epithelial surface expressed the ICAM-1 antigen more abundantly than undifferentiated control monocytes (FIG. 2A1). Dual labeling of the co-cultures for CD45 and ICAM-1 confirmed that adherent cells were monocytes/macrophages (FIG. 1D). The increased ICAM-1 expression on monocytes/macrophages after co-cultivation with A549 cells was confirmed at the population level with flow cytometry analysis since the mean ICAM-1 signal intensity of the CD45-positive cell fraction was significantly higher than that of undifferentiated monocytes, i.e. 1.3-fold (p=0.02). Although, the ICAM-1 signal of the U937 cells co-cultured with A549 epithelial cells in three-dimensional was lower (1.4-fold, p=0.002) than the ICAM-1 signal of PMA-differentiated U937 macrophages. Next, the functional activity of adherent U937 cells was assessed by testing phagocytic capabilities. Monotypic cultures of U937 monocytes could not phagocytose 2 μm fluorescent beads, while PMA-differentiated U937 macrophages displayed engulfment of one-to-multiple beads (FIG. 2B2-3), in agreement with literature (Whyte et al., 2000, Hsieh et al., 2007). FIGS. 2B1-3 show phagocytosis of 2 μm fluorescent beads by the three-dimensional A549-U937 co-culture (B1), compared to undifferentiated U937 cells (B2) and PMA-differentiated macrophages (B3). Macrophage-like cells in the three-dimensional co-culture (B1) are labelled with the CD45 antibody (orange, shown as 1) showing specific uptake of beads (white-green, shown as 1A) by this cell population. Both Z-stack assembly (B1) and single plane (indent B1) images are presented.

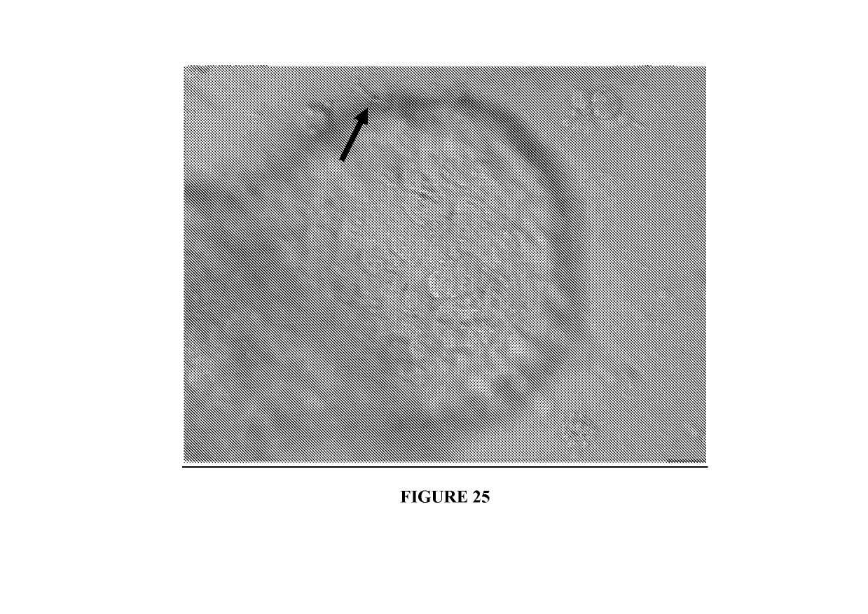

Surprisingly, U937 cells (CD45 positive) co-cultured with A549 cells in three-dimensional were capable of phagocytosing 2 μm beads (FIG. 2B1). No phagocytic activity was observed in three-dimensional A549 monotypic cultures, except for occasional adherence of beads to the epithelial surface. FIG. 25 shows a three-dimensional A549-U937 co-culture model infected with Pseudomonas aeruginosa PAO1. The arrow points out uptake of bacteria by phagocytic cell (image was derived from a movie where phagocytosis could be observed). These data indicate that macrophage-like activity was acquired by U937 monocytes when co-cultured three-dimensionally with A549 epithelial cells.

Effective Ratio of Macrophages to Epithelial Cells in the Three-Dimensional Co-Culture.

The effective ratio of CD45-positive macrophage-like cells to the total cell population was determined by flow cytometry, after 72 hours of co-cultivation. On average, 3.1±1.5% macrophage-like cells were found in the co-culture, corresponding to an effective ratio of 1 monocyte per 32 epithelial cells.

Response of Monotypic and Co-Cultures to 3-oxo-C12 HSL

Cell Viability.

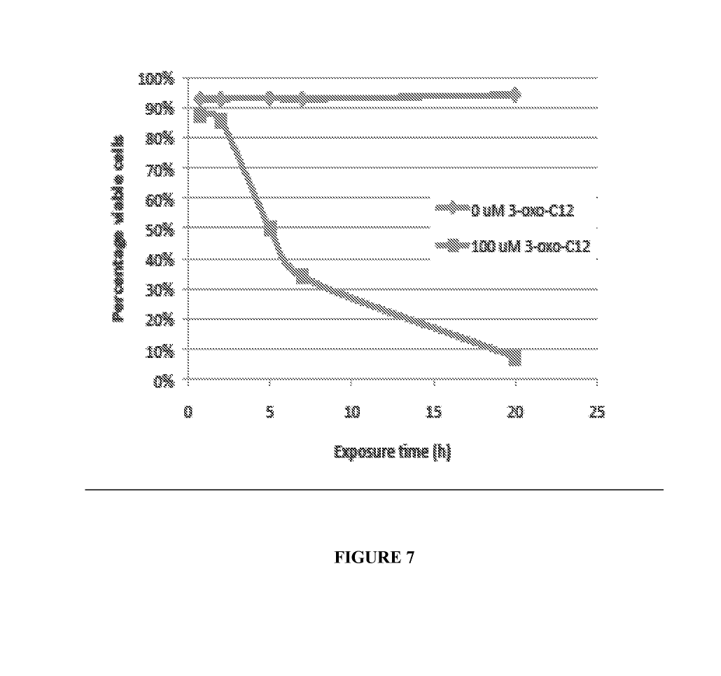

As presented in FIG. 7, pure cultures of U937 cells started to show significant loss of viability after 2 hours of exposure to 100 μM 3-oxo-C₁₂ HSL. After 5 hours of exposure, the viability decreased to 50%, and less than 10% viable cells were detected at the 20-hour time point (FIG. 7, Table 1). Lower concentrations of 3-oxo-C₁₂ HSL (5 μM and 10 μM) did not induce significant differences in monocyte cell viability (Table 1). Exposure of A549 three-dimensional monotypic cultures to 3-oxo-C₁₂ HSL did not induce cell death using concentrations of 5, 10 and 100 μM (Table 1), which was in agreement with previously published data (Tateda et al., 2003). When U937 cells were co-cultured with A549 epithelial cells in three-dimensional for up to 20 hours, these macrophage-like cells were minimally affected in viability following exposure to 100 μM 3-oxo-C₁₂ HSL (Table 1). Similarly, exposure of three-dimensional A549-U937 co-cultures to 5 and 10 μM 3-oxo-C₁₂ HSL did not induce increased death in the attached macrophage-like cell population when compared to the control. It should be noted that exposure to 100 μM 3-oxo-C₁₂ HSL decreased the ratio of U937 cells to the total cell population in three-dimensional A549-U937 aggregates by 37±5% when compared to the non-exposed control. Nevertheless, inclusion of all detached cells in a subsequent experiment indicated no difference in macrophage cell death between cultures exposed to 100 μM 3-oxo-C₁₂ HSL and the DMSO control. Epithelial cell viability in the three-dimensional A549-U937 culture was not affected following exposure to the tested concentrations of 3-oxo-C₁₂ HSL. The presence of porous microcarrier beads in the three-dimensional A549-U937 co-culture was not at the origin of the observed protection of U937 cells against the deleterious effects of 3-oxo-C₁₂ HSL, since U937 cultured with microcarrier beads showed a similar decrease in viability as monotypic U937 cells in the absence of microcarrier beads (Table 1).

TABLE 1
Percentage viability of A549 and U937 cells following 20 hour exposure to the
quorum sensing molecule 3-oxo-C₁₂ HSL. U937 cells were exposed alone (column
2), in the presence of microcarrier beads (column 3), and when co-cultured three-
dimensionally with A549 cells (column 6). A549 cells were exposed alone (column
4) and when co-cultured three-dimensionally with U937 cells (column 5). Error
bars represent the mean ± standard deviation (N = 3).

| | Cell type | | | | |
|---|---|---|---|---|---|
| | | | | A549-U937 3-D | |
| Exposure | U937 | U937 + beads | A549 3-D | A549 | U937 |
| DMSO (solvent control) | 88.7 ± 0.8 | 85.5 ± 1.0 | 97.5 ± 0.00 | 91.8 ± 0.02 | 97.0 ± 0.04 |
| 5 μM 3-oxo-C₁₂ HSL | 82.8 ± 0.2 | 89.1 ± 1.4 | 97.7 ± 0.00 | 90.3 ± 0.01 | 97.1 ± 0.01 |
| 10 μM 3-oxo-C₁₂ HSL | 75.6 ± 0.6 | 90.9 ± 0.4 | 97.8 ± 0.00 | 92.6 ± 0.00 | 99.3 ± 0.01 |
| 100 μM 3-oxo-C₁₂ HSL | 4.2 ± 0.5 | 5.3 ± 0.4 | 94.9 ± 0.02 | 84.3 ± 0.00 | 93.8 ± 0.03 |

Assessment of 3-oxo-C₁₂ HSL Concentrations as a Function of Time.

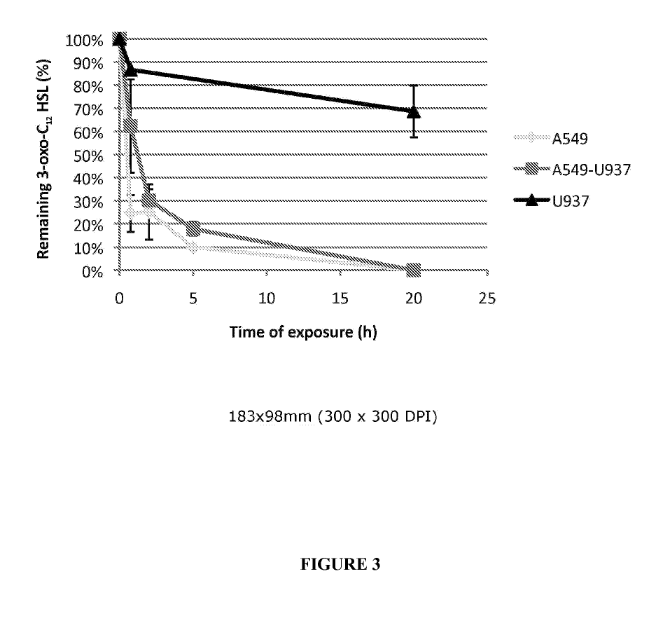

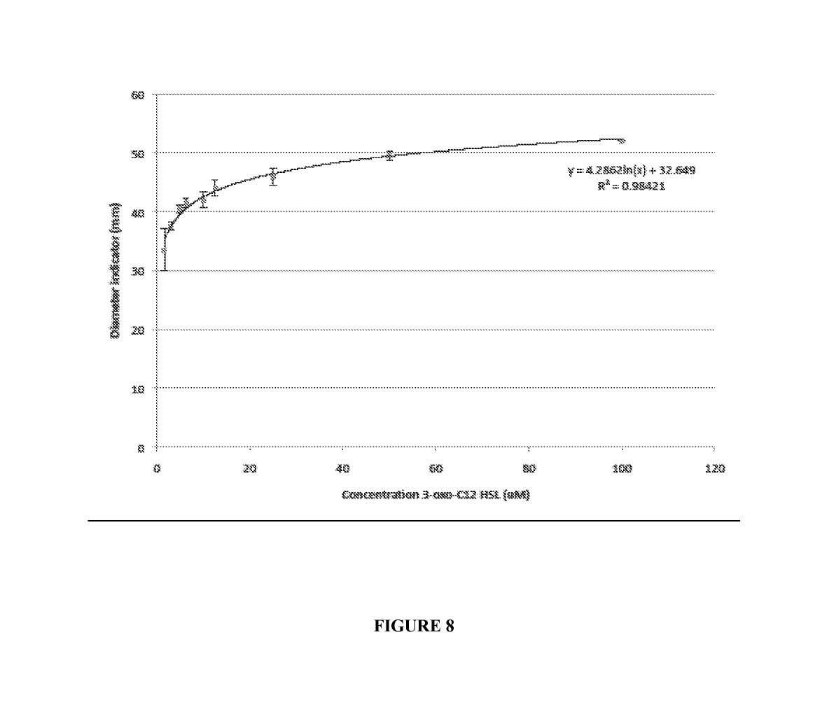

In three-dimensional monotypic (A549) and co-cultures (A549-U937), the concentration of 3-oxo-C₁₂ HSL rapidly decreased with time (FIG. 3). In FIG. 3, results are presented as the ratio of the remaining concentration to the original 3-oxo-C₁₂ HSL concentration (100 μM). Error bars represent the mean±standard deviation (N=2). After 2 hours of exposure, 30% of the original 3-oxo-C₁₂ HSL concentration remained, and no residual 3-oxo-C₁₂ HSL could be detected after 20 hours of exposure. Differences in 3-oxo-C₁₂ HSL concentrations between the three-dimensional A549 monotypic and three-dimensional A549-U937 co-cultures were not significantly different. Surprisingly, cultures of U937 cells exposed to 3-oxo-C₁₂ HSL showed minimal differences in 3-oxo-C₁₂ HSL concentration over time as compared to three-dimensional A549 and three-dimensional A549-U937 cultures, indicating that A549 cells caused the observed decrease in 3-oxo-C₁₂ HSL QS signal. A standard curve relating 3-oxo-C₁₂ HSL concentrations to the adopted plate assay is shown in SI (FIG. 8).

ICAM-1 Expression.

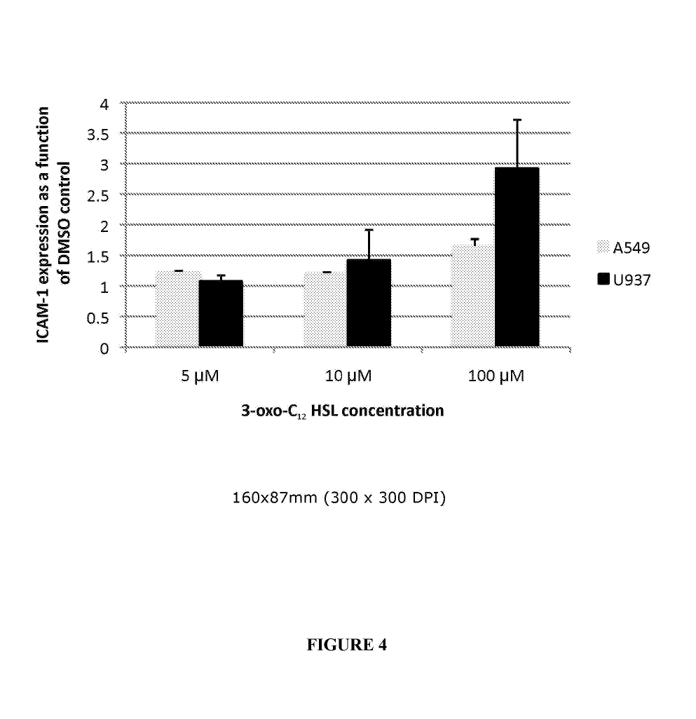

ICAM-1 expression of A549 cells (derived from the three-dimensional A549-U937 co-culture) exposed to 100 μM 3-oxo-C₁₂ HSL significantly increased when compared to the solvent control (1.66±0.11-fold, p<0.01) (FIG. 4). In FIG. 4, cultures were exposed for 20 hours to the QS molecules and analyzed with flow cytometry. Both cell populations were separated using CD45 as a specific marker for leukocytes. Error bars represent the mean±standard deviation (N=3).

Lower concentrations of the QS molecule did not affect ICAM-1 expression. Similar trends were observed with three-dimensional monotypic cultures of A549 cells. A significant increase (2.93±0.79-fold, p<0.01) in the ICAM-1 signal of the macrophage-like cells was observed when the three-dimensional A549-U937 co-culture was exposed to 100 μM 3-oxo-C₁₂ HSL, compared to the control samples (FIG. 4).

### Experimental Procedures

### Cell Lines, Culture Media and Growth Conditions

The alveolar epithelial cell line A549 (ATCC CCL-185) and the monocytic cell line U937 (ATCC CRL-1593.2) originated from the American Type Culture Collection (ATCC, Manassas, Va.). All eukaryotic cells were cultured in GTSF-2 medium (Hyclone, Logan, Utah) supplemented with 10% fetal bovine serum, 2.5 mg/l insulin transferring sodium selenite (Sigma-Aldrich), and 1 ml/l Fungizone (Invitrogen) and were incubated at 37° C. under 5% CO₂.

### Development of Three-Dimensional Monotypic and Co-Culture Models

Three-Dimensional A549 Monotypic Culture.

The three-dimensional A549 monotypic cultures were made as previously described (Carterson et al., 2005).

Three-Dimensional A549-U937 Co-Culture.

A549 three-dimensional aggregates were grown as described above and after 9 days of three-dimensional A549 cultivation, undifferentiated U937 monocytes were added to the HARV as follows. three-dimensional A549 cells were counted by trypsinization of an aggregate sample and U937 cells (grown in T75 flasks) were added in a 1:1 ratio to the three-dimensional A549 culture. A549 and U937 cells were co-cultured in the HARV at 20 rpm for 72 h. After addition of the monocytes, culture medium was changed after 48 hours of co-cultivation where after medium was replenished every 24 hours.

### 2-D Monolayers as Controls

To obtain differentiated macrophages, U937 monocytes were exposed to 10⁻⁸M PMA (originating from a 10⁻³M stock in DMSO) (Sigma-Aldrich) for 48 hours. Exposure of monocytes to phorbol esters induces morphological, physiological and molecular characteristics of terminally differentiated macrophages (Rovera et al., 1979, Prieto et al., 1994). For CLSM staining purposes, monocytes were differentiated with PMA and A549 cells were grown in 6-well plates containing sterile coverslips. For CLSM staining of undifferentiated U937, cytodex-3 beads (5 mg/ml) were added to the culture medium, allowing adhesion of U937 to the beads and facilitation of the staining procedure.

### Confocal Laser Scanning Microscopy

Antibodies.

The antibodies used for CLSM imaging in the present study were from mouse origin and targeted the human CD45 (Abcam), ICAM-1 (Abcam), MUC5AC (Invitrogen), ZO-1 (Invitrogen) and β-catenin (Chemicon). All primary antibodies were used in a dilution of 1:50. ICAM-1 was the only directly labeled antibody used for CLSM staining and was conjugated with phycoerythrin (PE). A goat anti-mouse secondary antibody labeled with Alexa Fluor 555 (Invitrogen) was used to detect the bound primary antibodies and was diluted 1:500 in blocking solution (8% bovine serum albumin, 0.05% Triton-X100 in DPBS). For dual labeling purposes of ICAM-1 and CD45 or MUC5AC, a rabbit anti-mouse secondary antibody was used conjugated with Alexa Fluor 488 (Invitrogen) to label CD45 and MUC5AC primary antibodies. Phalloidin conjugated with Alexa Fluor 633 (Invitrogen) was used to stain the F-actin fraction of the cytoskeleton. The cell nucleus was visualized with 4′,6-diamidino-2-phenylindole hydrochloride (DAPI) (Invitrogen).

Fixation and Staining.

three-dimensional aggregates of monotypic and co-culture models, undifferentiated U937 cells (adherent to microcarrier beads) and coverslips containing A549 monolayers or PMA-differentiated macrophages were rinsed 3 times with DPBS (Invitrogen) and fixed with 4% paraformaldehyde (in PBS) (Electron Microscopy Services) for 30 min at ambient temperature. Non-specific binding sites were blocked with blocking solution (8% bovine serum albumin, 0.05% Triton-X100 in DPBS) for 30 min. Cells were washed 3 times with Tween-PBS (T-PBS) (0.1% Tween 20 in DPBS) and incubated with the primary antibody (diluted in blocking solution) for 1 hour at room temperature. After rinsing 3 times with T-PBS, cells were incubated, when needed, with the secondary antibody (diluted in blocking solution) for 30 min in a darkened environment. Control samples were stained with the secondary antibody alone and indicated the absence of non-specific binding. For dual staining of cells with ICAM-1 and CD45 or MUC5AC, the above-mentioned protocol was first performed for the CD45 or MUC5AC primary and secondary antibody followed by staining with the directly labeled ICAM-1 antibody. For staining of the cytoskeleton, cells were incubated 30 min with phalloidin-Alexa Fluor 633 (Invitrogen). Aggregates or coverslips were mounted on a glass slide containing Prolong Gold with DAPI mounting solution (Invitrogen).

Imaging.

Optical sections of the three-dimensional aggregates and monolayers were obtained using a Zeiss LSM 510 Duo laser scanning microscope equipped with detectors and filter sets for monitoring emissions of the selected fluorophores. Images were acquired using a Plan-Neofluar 40×/1.3 oil DIC objective and were analyzed with the Zeiss LSM software package. Z-stacks of aggregates (200-300 μm) were made with a 1 μm interval and post-acquisition reconstruction of three-dimensional images was performed with the Zeiss LSM software package. Axiovision 4.7 software from Carl Zeiss was used to further process collected images.

### Flow Cytometry

Antibodies.

Mouse anti-human CD45 (Invitrogen) and ICAM-1 (Invitrogen) conjugated with phycoerythrin (PE)-Cy5 and FITC respectively were used for flow cytometry staining. Sample preparation and fluorescent staining of cells. Cells were rinsed 2 times with pre-warmed HBSS (Invitrogen), detached from microcarrier beads/culture flasks with 0.25% trypsin-EDTA (Invitrogen) and counted using a hemocytometer. For inclusion of cells detached from the microcarrier beads (three-dimensional A549 and three-dimensional A549-U937 co-cultures) in the frame of QS exposure studies (see below), whole cultures (both aggregates and bulk liquid) were centrifuged at 1200 rpm for 6 min for each washing step. Next, microcarrier beads were removed (if needed) by filtering through a stainless steel cell dissociation sieve (pore size 104 μm) (Sigma-Aldrich). Approximately 1-2×10⁶ cells were used for staining. Cells were washed in ice cold staining buffer (10% FBS, 1% sodium azide in DPBS), resuspended in 3% BSA (in DPBS) containing the antibody (concentrations according to manufacturer's instructions) and incubated at 4° C. in the dark. For dual labeling, the described protocol was consecutively repeated for each antibody. Subsequently, cells were rinsed 2 times with ice cold staining buffer and resuspended in fresh staining buffer for analysis.

Cell Viability Assay.

Assessment of live-, apoptotic-, and necrotic cell populations was performed with the annexin V-propidium iodide (PI) kit for flow cytometry analysis (ImmunoSource) according to the manufacturer's instructions.

Flow Cytometry Analysis.

Stained cell suspensions were analyzed with an EPICS XL flow cytometer (Beckman-Coulter) or a Cytomics FC 500 system (Beckman-Coulter), equipped with an argon laser and filters for adequate excitation of the chosen fluorophores. Forward scatter, side scatter, FITC-, PI and PE-Cy5 emissions were measured and a total of 10,000 cells were recorded for each sample. The respective cell populations were delimited to eliminate background signals originating from cell debris. To assess background fluorescent signals from the tested cell populations, non-stained samples were included.

Determination of the U937-A549 Ratio for Three-Dimensional Co-Cultures.

three-dimensional co-cultures of A549 and U937 cells (as described above) were stained for CD45, since this marker specifically stains leukocytes. The ratio of CD45-positive cells (U937) to the total cell population [i.e. CD45 positive and -negative cells (A549)] revealed the effective fraction of monocytes/macrophages in the co-culture.

Table 2 below presents the different variables that were tested for the successful generation of the three-dimensional A549-U937 co-culture model.

### Phagocytosis Assay

The phagocytosis activity of monocytes and macrophages cultured alone or co-cultured with A549 cells in three-dimensional was assessed based on the uptake of bacterial-sized 2 μm fluorescent polypropylene beads (Fluoresbrite plain YG, Polysciences). The phagocytosis assay was performed as previously described (Gao et al., 2010).

Exposure of Monocytes, Three-Dimensional Monotypic, and Three-Dimensional Co-Cultures to 3-oxo-C₁₂ HSL

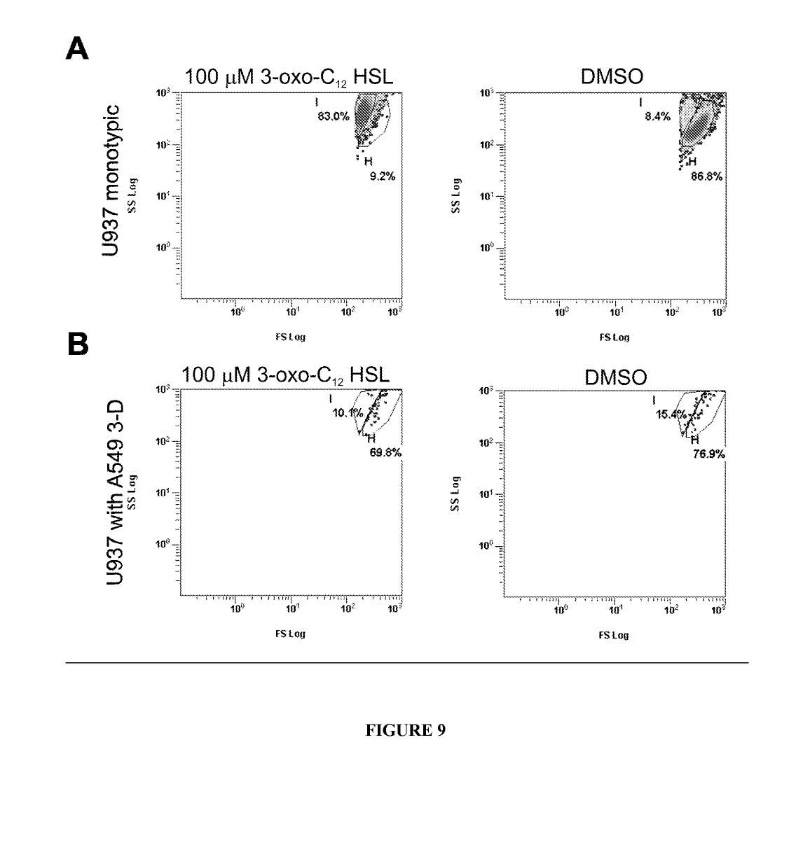

After 72 hours of co-cultivation, aggregates containing 2×10⁶ cells were seeded into 6-well plates containing fresh medium (5 ml total volume). Subsequently, aggregates were spiked with 5, 10 or 100 μM 3-oxo-C₁₂ HSL (Sigma-Aldrich) (starting from a 20 mM stock solution in DMSO, stored at −20° C.). Equal volumes of DMSO, containing the respective amounts of 3-oxo-C₁₂ HSL, were added to each well (25 μl) and a solvent control (25 μl DMSO) was included in each experiment. Six-well plates were placed at 37° C. in a 5% CO₂ environment and incubated for 20 hours. For exposure assays of monocytes to 3-oxo-C₁₂ HSL, 2×10⁶ U937 cells/well were transferred in 6-well plates. To assess the influence of 3-oxo-C₁₂ HSL on monocytes cultured on microcarrier beads, collagen I-coated beads were added to the cells at the same concentration as for seeding three-dimensional cultures (1 bead per 2500 cells). Subsequently, monocyte cultures (with or without microcarrier beads) were spiked with 3-oxo-C₁₂ HSL (see above). A solvent control was included. After 20 hours incubation, cells were processed for flow cytometry analysis. Cell viability was assayed with trypan blue (0.4%) (Sigma) and/or with the annexin V-PI kit (ImmunoSource) (see above). However, since both annexin V-PI and trypan blue staining indicated that cell death was associated with an alteration in cell size, as reflected through the forward scatter signal of the flow cytometer (FIG. 9), cell size was concluded to be an appropriate indicator of cell death. FIG. 9A shows representative flow cytometry charts of U937 monocytes exposed to 100 μM 3-oxo-C₁₂ HSL and to the solvent control DMSO. FIG. 9B are examples of the three-dimensional A549-U937 co-culture model exposed to 100 μM 3-oxo-C₁₂ HSL and to the solvent control DMSO. The presented cell population is gated for CD45 positive cells and thus only includes monocytes/macrophages. For FIGS. 9A-B, “I” represents the percentage of dead cells, while “H” is the viable cell population. Consequently, cell viability was further assessed based on the shift in forward scatter signal (see Table 1).

Assessment of 3-oxo-C-₁₂ HSL Concentrations

Following exposure of three-dimensional cultures and monocytes to 3-oxo-C₁₂ HSL, cell-free samples were taken and stored at −20° C. at indicated time points. The remaining 3-oxo-C₁₂ HSL concentration was assessed as previously described (Crabbé et al., 2008) using the indicator strain E. coli harboring the reporter plasmid pUCP22NotI-PlasB::gfp(ASV)Plac::lasR (Hentzer et al., 2002). A 3-oxo-C₁₂ HSL standard series was included ranging from 1.63 to 100 μM. Culture medium containing microcarrier beads was used as a control to normalize for bioadsorption.

### Statistics

All experiments were performed at least in biological duplicate and technical triplicate. The statistical determination of significance (α=0.05) was done with Microsoft Office Excel 2003 using a two-sample Student t-test on the biological repeats of each experimental condition.

Epithelial ICAM-1 Expression Following Infection with P. Aeruginosa

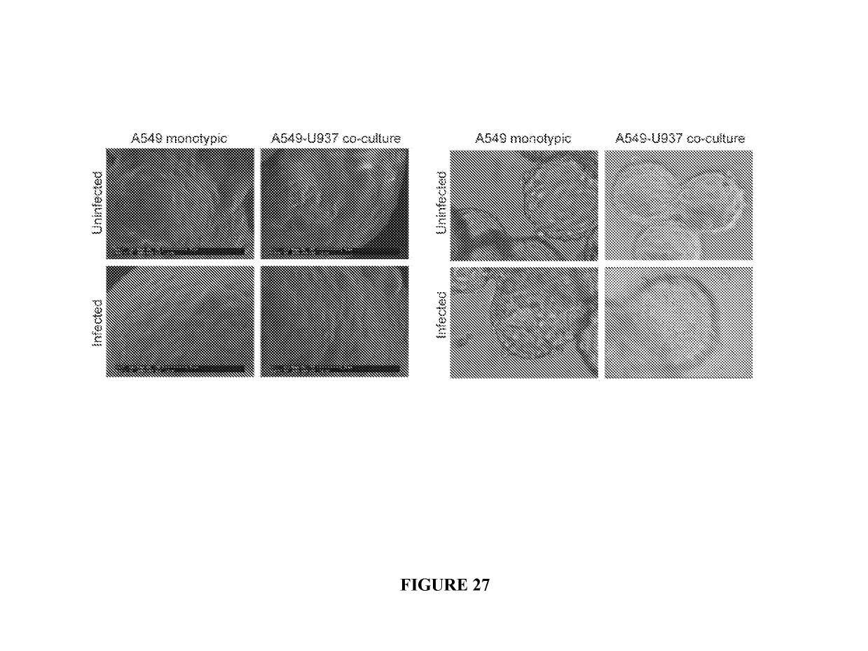

3-D monotypic and co-cultures were subjected to P. aeruginosa PAO1 infection after 72 hours of co-cultivation. After 20 hours of infection, the bacterial load in both culture types was approximately 1.4×10⁸ CFU/ml. FIG. 27 shows light microscopic- and SEM analysis indicated that a majority of the aggregates remained intact post-infection (i.e. cells did not detach from the microcarrier beads).

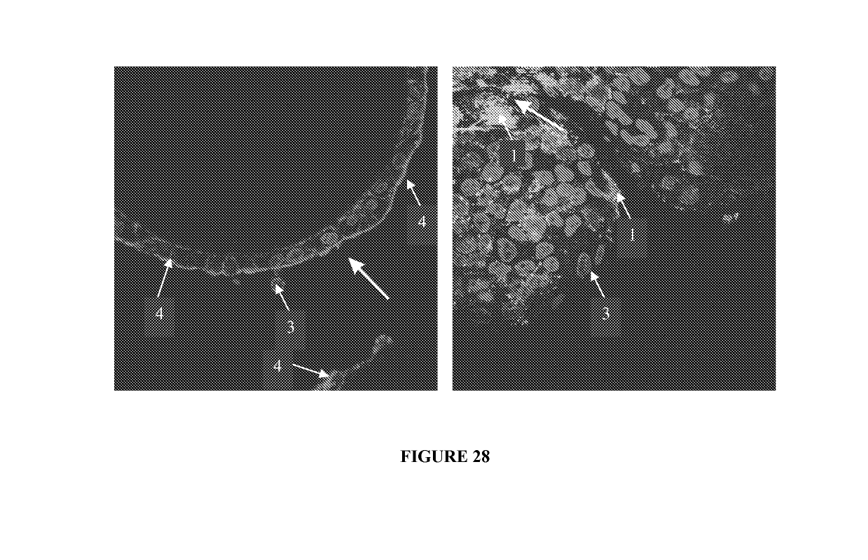

CLSM imaging revealed that most of the detected P. aeruginosa cells were present in the mucus layer (based on MUC5AC staining) and did not invade the epithelial cells (FIG. 28). FIG. 28 shows localization of P. aeruginosa PAO1 in the infected A549-U937 co-culture. Bacteria did not invade host cells (A) and remained in the epithelial mucus layer (B). Arrows indicate GFP-labeled bacteria (green, shown as 2), orange (shown as 1) staining reflects MUC5AC, the cytoskeleton is labeled in red (shown as 4) and cell nuclei are in blue (shown as 3). However, it should be noted that only a few bacterial cells could be visualized after MUC5AC staining for confocal imaging. The experimental procedures, involving many washing steps, could be at the origin of the accidental removal of adherent bacteria. Indeed, when aggregates were immediately emerged in Prolong Gold mounting solution containing DAPI post-infection and visualized, an increased number of bacteria could be observed (data not shown). Epithelial cell viability was estimated post-infection with the trypan blue exclusion assay and indicated that in all infected cultures, less than 10% cell death occurred.

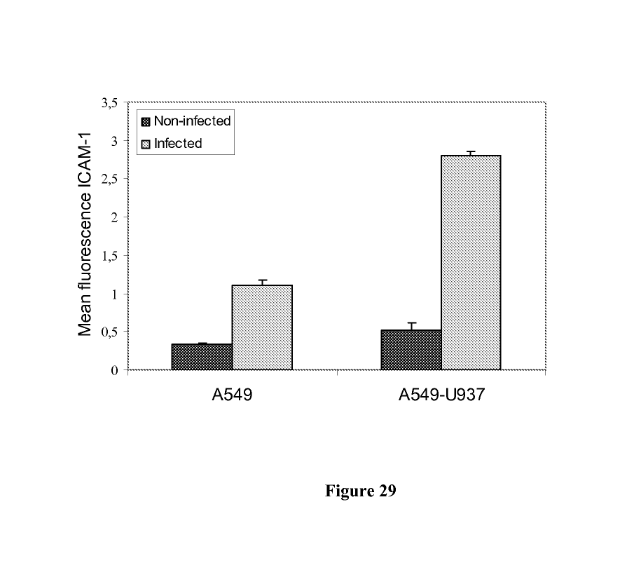

ICAM-1-expression was enhanced in both monotypic and co-cultures following infection, when compared to non-infected controls. While the non-infected A549-U937 cultures only displayed a slightly higher, non significant, ICAM-1 expression than the A549 cultures (cf. ICAM-1 after 4 days of co-cultivation, figure not shown), the increase in ICAM-1 signal intensity after infection was 1.6-fold higher in the co-cultures compared to the monotypic cultures (p<0.05) (FIG. 29). FIG. 29 shows mean ICAM-1 fluorescence in A549 monotypic and A549-U937 co-cultures before and after 20 hours infection with P. aeruginosa PAO1. Remarkably, after 20 hours of infection, no adherent mononuclear phagocytes could be found in the co-culture. The latter result could be due to death of monocytic cells caused by infection or could be explained by the detachment of the monocytes/macrophages from the mucus layer to fight infection.

### Cytokine Production in Response to P. Aeruginosa Infection

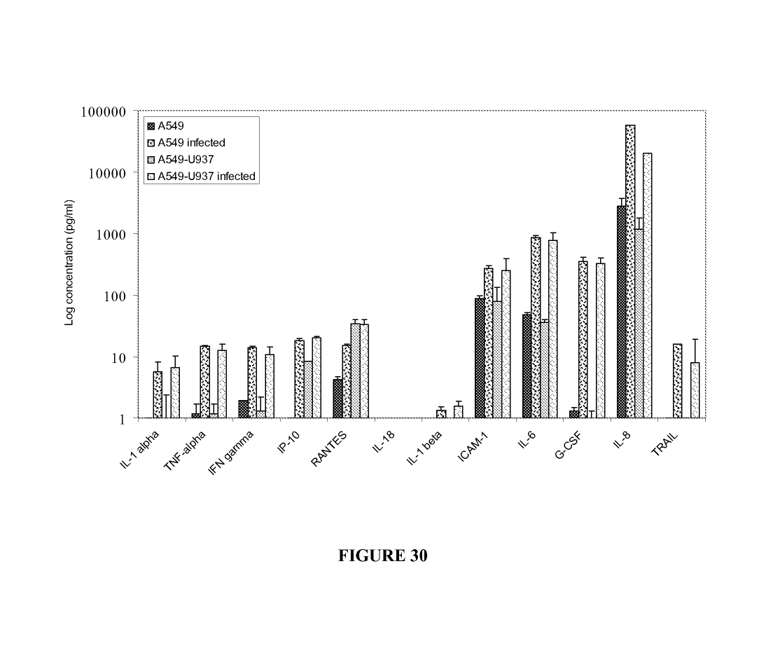

Prior to infection with P. aeruginosa, both monotypic and co-cultures produced background levels of the tested cytokines except for RANTES, IP-10, ICAM-1, IL-6 and IL-8 (FIG. 30). FIG. 30 shows cytokine concentrations of 3-D A549 monotypic and A549-U937 co-cultures before and after exposure to P. aeruginosa PAO1. While each experiment was performed in biological duplicate, the IL-8 concentration for one biological sample of both monotypic and co-cultures could not be monitored since it was out of the detection range of the Bioplex-200 device (upper limit). The IL-8 data could thus not be processed for statistical analysis. A majority of the tested cytokines were induced upon infection with P. aeruginosa, except for IL-18 which remained at background levels (FIG. 30). The fold-increase in cytokine concentration after 12 hours of infection of monotypic and co-cultures was not significantly different for most of the assayed cytokines. Only IP-10 and RANTES showed a 27.8-fold and a 4.1-fold higher induction respectively in the monotypic A549 culture when compared to the A549-U937 co-culture (p<0.05). This observation might however be ascribed to a higher level of RANTES and IP-10 in the non-infected A549-U937 cultures. Indeed, cytokine profiling of monotypic U937 cultures indicated that these cytokines were amongst the highest produced in culture (data not shown).

### Discussion

The simultaneous presence of alveolar epithelium and functional macrophages in an in vitro model of alveolar tissue was important to adequately investigate the response of the lung to bacterial virulence factors. The RWV technology was adopted to develop a three-dimensional organotypic co-culture model of alveolar epithelium and macrophages. This three-dimensional RWV-derived co-culture model of epithelium and immune cells showed the presence of naturally differentiated (i.e. no chemical treatment) macrophage-like cells in the mucus layer of the alveolar epithelium, as evidenced by their increased expression of the macrophage surface marker ICAM-1 and enhanced phagocytosis of bacterial-sized beads, when compared to monocytes cultured alone. These data indicated that the adopted culture conditions, in which monocytes were placed in contact with highly differentiated alveolar epithelium, induced the spontaneous transition to a phenotype with characteristics of normal alveolar macrophages. Indeed, contact of monocytes with the extracellular matrix, epithelial cells and their secreted factors play a role in their in vitro differentiation to macrophages (Spottl et al., 2001, Striz et al., 2001, Spoettl et al., 2007). Co-cultivation of monocytes with alveolar epithelium in three-dimensional also increased the expression of ICAM-1 on the cell surface of the alveolar epithelial cells.

These data confirmed the role of alveolar macrophages in the mediation of the epithelial innate immune response, since ICAM-1 activates the phagocytic activity of macrophages (Paine et al., 2002). Consequently, the effective ratio of mononuclear cells to alveolar epithelial cells in the non-infected lung is important for the basal ICAM-1 level of the epithelium. The effective ratio of macrophages-to-alveolar epithelial cells in the three-dimensional co-culture models was determined to be on average 1:32 after 3 days of co-cultivation, comparable to the healthy non-infected lung (one alveolar macrophage to approximately 40 epithelial cells) (Crapo et al., 1983, Paine et al., 2002).

The secreted mucin, MUC5AC, presumably played a role in the adherence of the macrophage-like cells to the surface of the alveolar epithelium. These results are consistent with the architecture of the parental tissue in vivo, since alveolar macrophages are localized in the alveolar surfactant film lining the alveolar epithelium (Jonsson et al., 1986). The presence of a well-defined mucus layer on the surface of the alveolar epithelium reflects more closely the in vivo situation of patients with chronic obstructive pulmonary diseases, such as cystic fibrosis, since hypersecretion and reduced clearance of mucus in these patients results in the presence of a thick, viscous mucus layer in both the conductive and respiratory zones of the lung (Foweraker, 2009).

While this and other studies have shown that 3-oxo-C₁₂ HSL induced apoptosis in pure cultures of monocytes and macrophages at concentrations above 25 μM (Tateda et al., 2003, Kravchenko et al., 2006, Li et al., 2009), exposure of the three-dimensional co-cultured monocytes/macrophages with alveolar epithelium to concentrations up to 100 μM did not induce cell death in either of the cell populations. Thus, this Example shows that alveolar epithelial cells can protect monocytes/macrophages against the detrimental effects of 3-oxo-C₁₂ HSL. Although not bound by any theory, the protection of macrophages in the three-dimensional co-culture model presumably occurred through enzymatic degradation of the QS molecules by the alveolar epithelium (Chun et al., 2004, Ozer et al., 2005). This Example indicates that alveolar epithelium removes 3-oxo-C₁₂ HSL from the cell environment before it has time to exert any cytotoxic effects on macrophages. This Example further suggests that the initial exposure of macrophages to 3-oxo-C₁₂ HSL, i.e. before removal by the A549 cells, did not trigger mechanisms of apoptosis or necrosis in the former population.

The tested 3-oxo-C₁₂ HSL concentrations were in a physiologically relevant range since P. aeruginosa biofilms, typically found in the lung mucus of cystic fibrosis patients, can locally produce 3-oxo-C₁₂ HSL concentrations up to 600 μM (Charlton et al., 2000), while planktonic cultures produce 3-oxo-C₁₂ HSL concentrations from 1 to 10 μM. This Example indicates that, due to protection by the alveolar epithelium, macrophages should still be able to trigger host immune responses, e.g. by means of ICAM-1 induction, despite the presence of high concentrations of 3-oxo-C₁₂ HSL. Indeed, this Example demonstrated that 3-oxo-C₁₂ HSL enhanced the expression of ICAM-1 in both alveolar epithelial and macrophage-like cells. In accordance with these results, Wagner and colleagues reported that the ligand of ICAM-1, CD11b, was induced in response to 3-oxo-C₁₂ HSL in polymorphonuclear neutrophils (Wagner et al., 2007). The 3-oxo-C₁₂ HSL molecules influence mammalian gene expression through, among others, selective impairment of the NF-κB pathway and phosphorylation of p38 and eIF2α protein kinases (Kravchenko et al., 2006, Kravchenko et al., 2008). Although not bound by any theory, since p38 has been demonstrated to mediate the expression of ICAM-1 in macrophages (Cui et al., 2009), the activation of the p38 pathway by 3-oxo-C₁₂ HSL was presumably at the origin of the observed increase in ICAM-1.

In conclusion, this Example developed an advanced three-dimensional multi-cellular alveolar co-culture model of epithelium and macrophage-like cells that displayed physiologically relevant morphological characteristics. These in vivo-like hallmarks included (i) highly differentiated alveolar epithelium, (ii) the autonomous differentiation of monocytes to functional macrophage-like cells, (iii) the localization of macrophage-like cells on the alveolar surface, and (iv) a macrophage-to-epithelial cell ratio of approximately 1:32.

This tissue system was used to investigate the host response to the QS molecule 3-oxo-C₁₂ HSL, allowing the profiling of key aspects of the complex networking between the epithelium and the prominent innate immune cell of the lung. We demonstrated for the first time that the deleterious effects of the 3-oxo-C₁₂ HSL QS signal of P. aeruginosa on mononuclear phagocytes could be counteracted by three-dimensional co-cultivation with alveolar epithelium and could trigger mechanisms of innate immunity in both cell populations.

Collectively, these data indicate that different cell types in the lung orchestrate the overall response of each single cell type to bacterial virulence factors; these interactions need to be taken into careful consideration when predicting the host response to infection with regard to potential therapeutic applications. Furthermore, this study stressed the importance of highly differentiated multi-cellular organotypic models to assess overall organ homeostasis and disease development.

### Example 2

### Three-Dimensional Co-Culture of Intestinal Epithelial Cells and Monocytes (U937)

The studies described in Example 2 established and characterized a biologically relevant functional three-dimensional co-culture model using human intestinal epithelial cells cultured with macrophages.

### Establishment of Organotypic Co-Cultures

Cell-Line Derived Model:

Three-dimensional co-culture models derived from human cell lines with i) large intestinal epithelial cells (HT29 colonic cells) and monocytes (U937), and with ii) small intestinal epithelial cells (Int-407) and monocytes were established and optimized. Extensive optimization parameters empirically tested and outcomes determined to date for this three-dimensional co-culture model development are shown in Table 2. Separate 3-D monotypic cultures of each of these cell lines were also established as controls for the co-culture model. For the parameters listed in Table 2, sample aliquots of three-dimensional cultures (monotypic and co-culture models) were removed from the bioreactor at the times indicated below and monitored for i) kinetics of aggregate formation (using light microscopy), and ii) conditions for addition of macrophages to the intestinal cells using different macrophage-specific markers and differentiation for cell-specific markers (confocal microscopy) throughout the course of development of this model at the indicated timepoints to determine optimal time for harvesting. Once a procedure was established with the HT29 and U937 co-culture model, this procedure was also used to develop the small intestinal (Int-407) co-culture model with U937 cells.

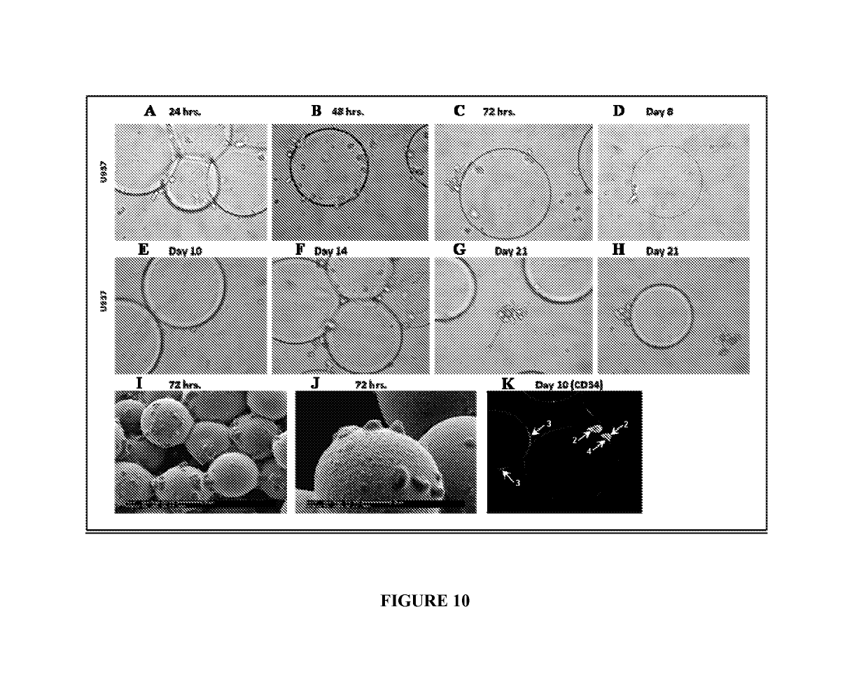

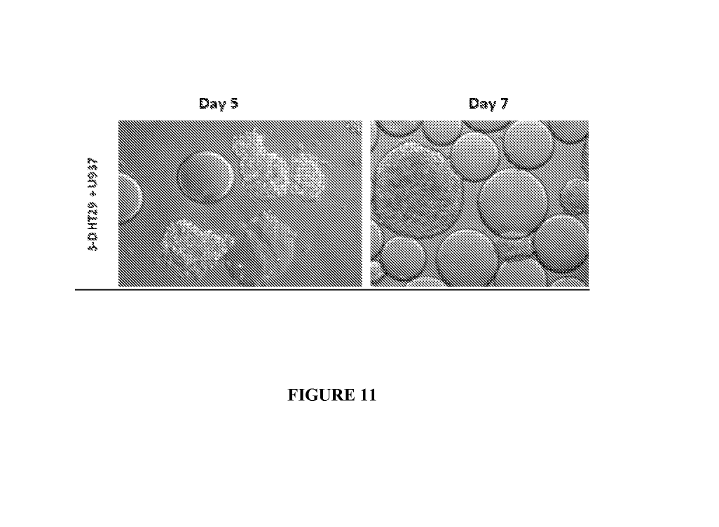

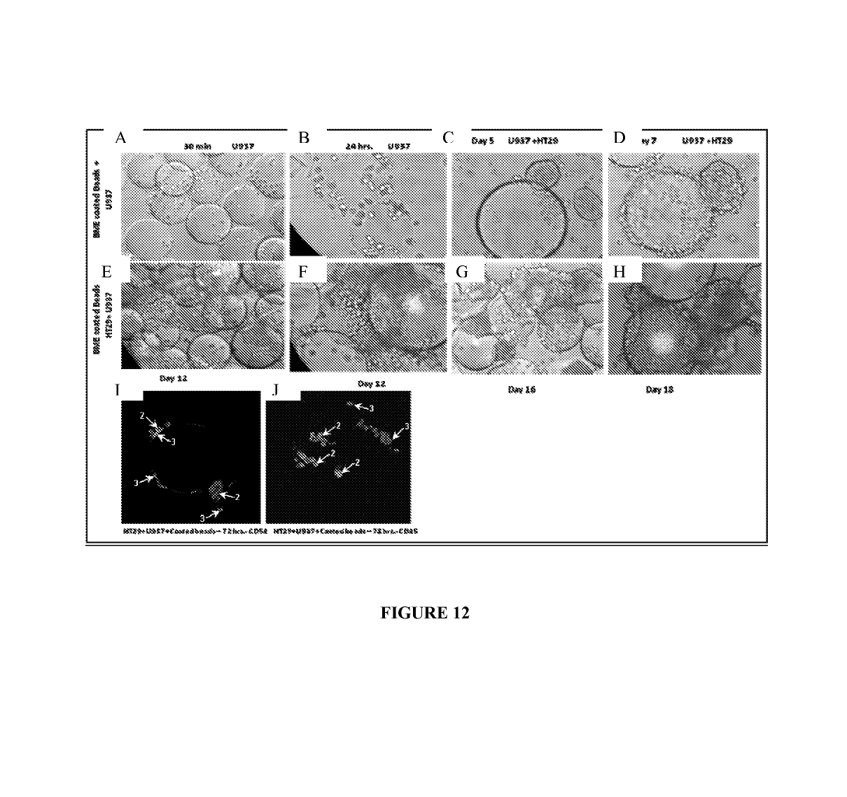

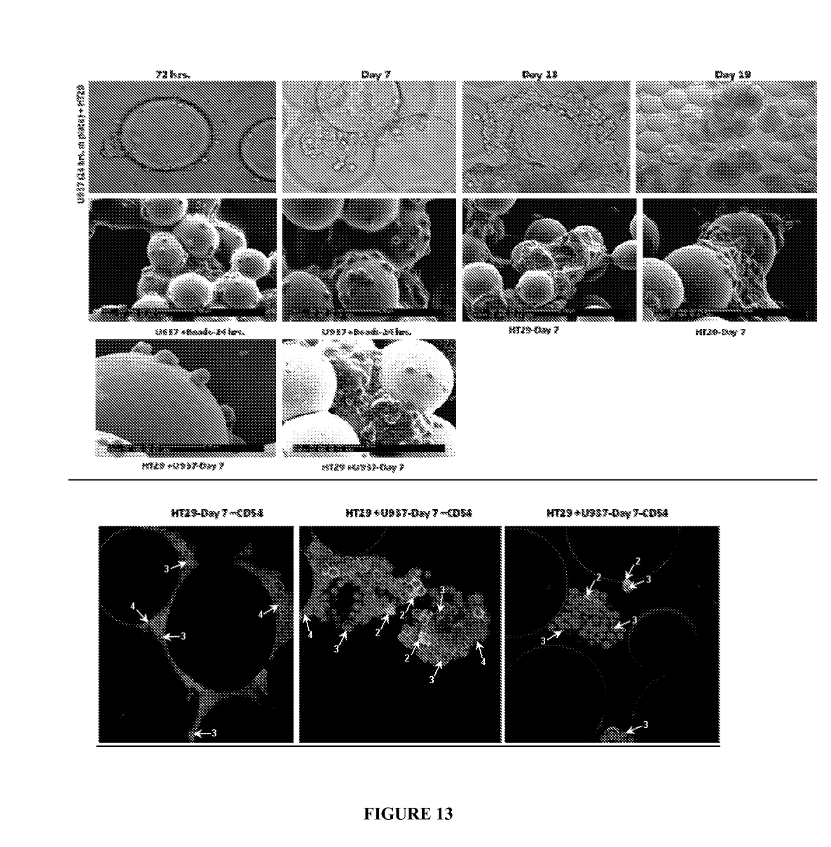

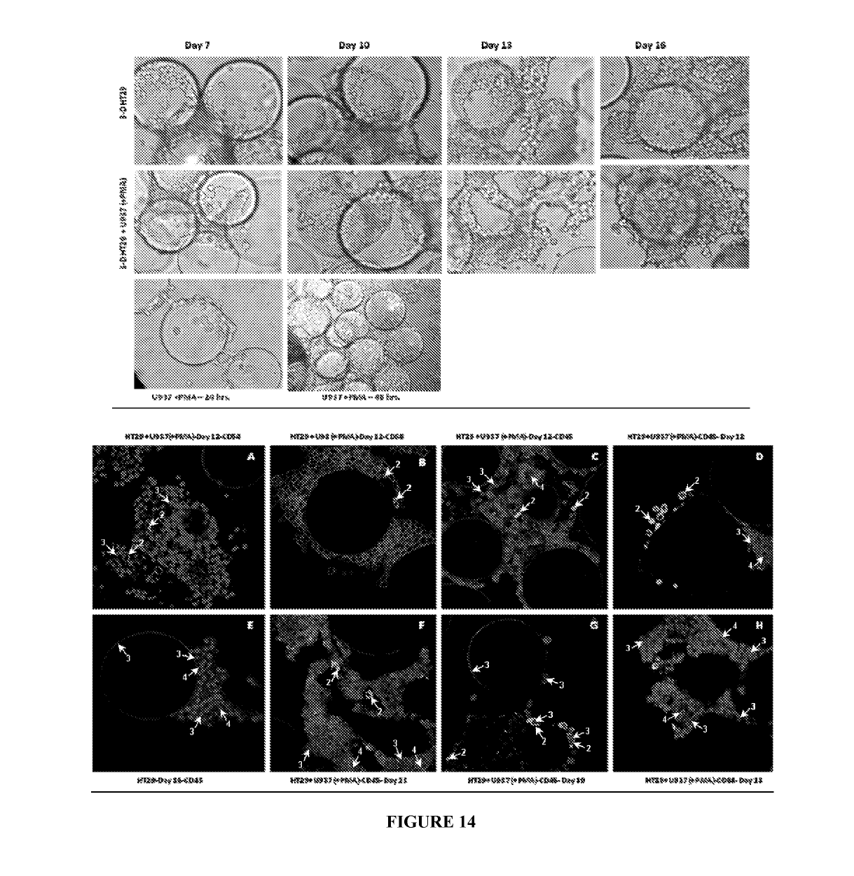

TABLE 2
Optimization parameters empirically determined for
3-D intestinal co-culture model establishment

| Parameter | Conditions tested | Result |
|---|---|---|
| 1) Production of | Based on the paper by Lorkowski et | Due to the low fluid shear in the |
| Extracellular | al. J Immunol 2008, 180: 5707-5719, | bioreactor, the majority of the |
| Matrix (ECM) for | it has been shown that monocytes | U937 cells detached from the |
| co-culture model | and macrophages express almost all | beads, but those cells that |
| development | known collagen, especially Type | remained attached formed long |
| | VIII collagen, in abundance | extensions and appear quite |
| | depending on their stage of | differentiated (FIG. 10). The |
| | differentiation and cell density. To | increased expression of |
| | generate an ECM for epithelial cell | CD54/ICAM1 (Inter-cellular |
| | binding and also to determine the | Adhesion Molecule 1) after 72 |
| | differentiation pattern in a bioreactor, | hours in the bioreactor suggests the |
| | monocytic cell line U937 were | differentiation of these monocytes |
| | initially grown in T25 flasks and then | to macrophages. |
| | about 1 × 10⁷ cells were added to | |
| | Cytodex 3 microcarrier beads in the | |
| | RWV bioreactor. These cells were | |
| | grown in the bioreactor for 23 days. | |
| 2) Optimization of | a) Using the concepts of the previous | a) Flow cytometry was performed |
| timing of addition | experiment, U937 cells were grown | at the end of 21 days to quantitate |
| of monocytic cells | in the STLV in the presence of | U937 percentage in the aggregates. |
| (U937) | microcarrier beads for 72 hours. At | About 0.7% incorporation was |
| | the end of 72 hours, colonic | determined; further optimization is |
| | epithelial cells (HT29) were added to | contemplated. |
| | the U937 cells. The co-culture was | b) The cells did not adhere to the |
| | continued for 14 days, after which | beads and instead formed large |
| | the coc-ulture was transferred to a | stacked cellular aggregates that |
| | disposable HARV and continued for | appeared more like tumor models |
| | another 7 days. | (FIG. 11). The process of cell |
| | b) Both U937 and HT29 cells were | attachment and differentiation was |
| | added simultaneously to the | monitored by removing cells and |
| | microcarrier beads and grown in the | imaging on particular days. Model |
| | bioreactor. | was terminated on day 7. |
| 3) Coating Beads | After autoclaving the Cytodex I | Within 30 minutes of adding U937 |
| with different | microcarrier beads, they were coated | cells, the cells covered the coated |
| ECMs (MaxGel | with the MaxGel Human ECM | beads, suggesting that this ECM |
| Human ECM) | (provides a rich three-dimensional | enabled better attachment of the |
| | environment to promote cellular | monocytes (FIG. 12). This |
| | proliferation and contains | approach resulted in robust three- |
| | extracellular matrix components | dimensional aggregate co-culture |
| | including collagens, laminin, | formation with natural |
| | fibronectin, tenascin, elastin, and a | differentiation of monocytic cells, |
| | number of proteoglycans and | as shown by the strong expression |
| | glycosaminoglycans) in the presence | of macrophage markers e.g. CD54 |
| | of GTSF-2 and HBSS medium. U937 | and CD45 (FIG. 12). |
| | cells were added to these coated | |
| | beads in 6-well plates, and the plates | |
| | incubated at 37° C. for 24 hours. The | |
| | cells bound to beads were then added | |
| | to HT29 cells and grown in the | |
| | bioreactor. | |
| 4) Optimal media | Media types tested: | GTSF-2 (without Penn/Strep and |
| to use in co- | Monocytes: a) RPMI media with | Fungizone) was compatible for 3- |
| culture model | heat-inactivated serum (for initial | D co-culture model establishment |
| establishment | growth of U937 cells (which | and supported differentiation of |
| (HT29 colon cells | proliferate very well in this media) | both cell types. |
| and U937 | before switching to GTSF-2); b) | |
| monocytes) | GTSF-2 with and without Pen/Strep; | |
| | c) GTSF-2 with and without | |
| | Fungizone. | |
| | Colon cells: a) GTSF-2 without | |
| | Penn/Strep and Fungizone | |
| 5) Optimization of | a) U937 cells were grown in 6-well | a) Addition of U937 to beads in 6- |
| different growth | plates for 24 hours at 37° C. in the | well plates after 24 hours resulted |
| conditions | presence of Cytodex 3 microcarrier | in production of intact ECM (see |
| | beads. After 24 hours, cells bound to | SEM images), thus contributing to |
| | beads were gently removed from | the structural integrity of the |
| | plates and added to HT29 cells in a | underlying matrix (FIG. 13). |
| | bioreactor. | While this process yielded robust |
| | b) U937 cells were grown in 6-well | 3-D aggregate co-culture formation |
| | plates in the presence of Phorbol | with expression of macrophage |
| | Myristate Acetate (PMA) for 48 | markers (FIG. 13), flow |
| | hours. The differentiated cells were | cytometry data revealed a low |
| | gently removed from the plates and | percentage of U937 incorporation. |
| | grown together with HT29 cells in | This model was further improvised. |
| | the bioreactor. | b) This approach yielded |
| | | representative three-dimensional |
| | | co-culture models that self- |
| | | assembled into aggregates |
| | | containing both colonic epithelial |
| | | cells and macrophages as |
| | | determined by extensive |
| | | immunohistochemical profiling |
| | | (FIG. 14). Differential |
| | | expression of CD45, CD54, CD68 |
| | | and CD84 markers confirmed |
| | | macrophage integration in this co- |
| | | culture model. Flow cytometry |
| | | will also be performed to |
| | | quantitate cell type percentages in |
| | | the aggregates. |
| 6) Differentiation | A) Times profiled via light & | In general, expression patterns of |
| profiling | confocal microscopy: | cell specific markers in three- |
| | 3-D colon cells: days 7, 10, 13, and | dimensional co-culture models |
| | 16. | revealed tissue organization and |
| | Macrophages: 24 and 48 hours (light | differentiation relevant to that |
| | microscopy) | found in the normal tissue in vivo, |
| | 3-D co-cultures (colon cells + | as compared to the same cells |
| | macrophages): days 7, 10, 13, 16 and | grown as monotypic three- |
| | 19 | dimensional cultures. |
| | B) Differentiation and macrophage | |
| | markers tested: | |
| | Tight junction marker: ZO-1 | |
| | Monocyte/Macrophage markers: | |
| | CD54, CD45, CD68, CD84 | |
| | Mucus production: MUC5AC | |
| 7) Phagocytosis | A) Uptake of fluorescent beads | Both of these approaches tested |
| experiments | The phagocytosis activity of | positive for phagocytosis, thereby |
| | macrophages co-cultured with HT29 | confirming the functionality of the |
| | cells in 3-D was assessed | co-culture. The PMA- |
| | based on the uptake of bacterial-sized | differentiated U937 macrophages |
| | 2 mm fluorescent polypropylene | in the presence of the HT29 cells |
| | beads. | displayed engulfment of one-to- |
| | B) Uptake of GFP labeled bacteria | multiple beads or several bacteria, |
| | The phagocytosis activity of U937 | but the monotypic HT29 culture |
| | differentiated macrophages co- | showed no such activity other than |
| | cultured with HT29 cells was | occasional attachment of beads to |
| | assessed based on the uptake of the | the surface. |
| | GFP labeled bacteria after 1 hour of | |
| | invasion. | |

### Results

FIGS. 10A-K shows generation of an extracellular matrix (ECM) by monocytes through light microscopy, SEM, and confocal images of U937 cells attached to microcarrier beads and differentiating over a period of time in a bioreactor. The process of cell attachment and differentiation was monitored by imaging on particular days. FIG. 10A-H shows images of U937 cells removed from the bioreactor at 24, 48, and 72 hours, as well as Days 8, 10, 14 and 21, respectively. FIGS. 10I-J show SEM images of the cells attached to beads after 72 hours. to check ECM formation. FIG. 10K shows confocal imaging to check cell differentiation using monocyte/macrophage differentiation marker, CD54. In FIG. 10K, cell nuclei are stained blue (DAPI) (shown as 3), the cytoskeleton red (shown as 4), and CD54 (ICAM-1) is stained green (shown as 2). The increased expression of CD54/ICAM1 (Inter-cellular Adhesion Molecule 1) after 72 hours in the bioreactor suggests the differentiation of these monocytes to macrophages.

FIG. 12A-J shows the results of coating beads with MaxGel ECM. The process of aggregate formation of these cultures was monitored by imaging at particular days using different magnifications. FIGS. 12A-J are light microscopy images of the kinetics of U937 cell attachment to beads (12A-B), 3-D aggregate formation by colon (HT29) and monocytic (U937) cells grown as 3-D co-cultures (12C-D), 3-D co-cultures (12E-H) removed from the bioreactor at days 5, 7, 12, 16, and 18, and confocal images of 3-D co-cultures showing expression of CD45 and CD54 monocyte/macrophage markers (12I-J). In FIGS. 12I-J, cell nuclei are stained blue (shown as 3, DAPI), and CD45 and CD54 (ICAM-1) green (shown as 2). Within 30 minutes of adding U937 cells, the cells covered the coated beads, suggesting that coating with ECM components enabled better attachment of the monocytes. This approach resulted in robust three-dimensional aggregate co-culture formation with natural differentiation of monocytic cells, as shown by the strong expression of macrophage markers, e.g. CD54 and CD45.

FIG. 13 shows the optimization of different growth conditions: U937 cells were grown in 6 well plates in the presence of beads for 24 hours before adding them to HT29 cells and growing both cell types in a bioreactor. The process of aggregate formation of these cultures was monitored by imaging at particular days using different magnifications. FIG. 13 shows images of 3-D cells in co-culture removed from the bioreactor on Days 3, 7, 13 and 19. The same days were profiled for 3-D monotypic cultures of HT29 cells. Increased expression of ICAM1 (CD54) suggests monocytic cells differentiating into macrophages. In the confocal image, cell nuclei are stained blue, (shown as 3 ((DAPI), the cytoskeleton red (shown as 4), and CD54 (ICAM-1) green (shown as 2).

Validation of the 3-D Co-Culture Model:

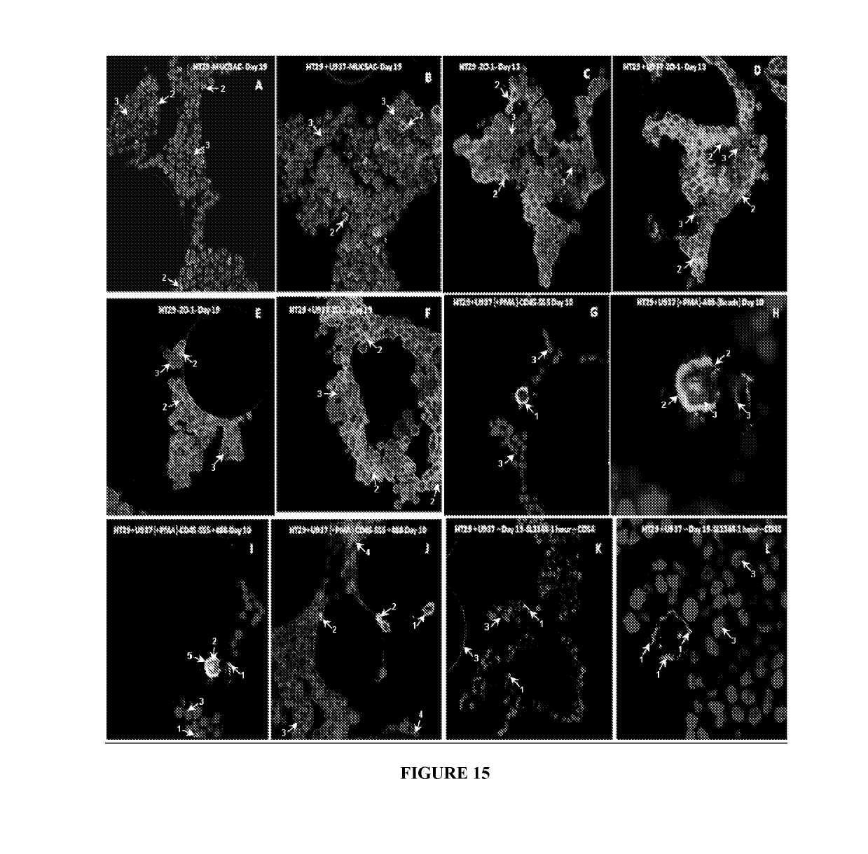

Models were characterized by a) light microscopy for kinetics of 3-D aggregate formation (FIG. 14), and b) immunohistochemical profiling using antibodies against cell-specific markers of differentiation characteristic for each respective cell type in vivo (FIGS. 14 and 15). FIG. 14 shows optimization of different growth conditions: U937 cells were grown in 6 well plates in the presence of beads and PMA (Phorbol myristate acetate) for 48 hours to allow differentiation of monocytes to macrophages before adding them to HT29 cells and growing both cells in a bioreactor. Cells from the 3-D monotypic and co-culture were removed from the bioreactor on Days 7, 10, 12, 13 and 16. Immunohistochemical profiling of colon (HT29) and macrophages (U937) cells grown as 3-D co-cultures (A-D, and F-H), 3-D monotypic culture (E). Confocal images of cultures from various timepoints were stained with antibodies against ICAM-1 (CD54) (A), CD68 (B), CD45 (C,D, E, F, G), and CD84 (H). Major differences in expression and distribution patterns of these monocytic/macrophage markers were observed between 3-D aggregates with 3-D co-cultures exhibiting expression relevant to that found in normal tissues. Data are from two experiments and are representative of independent batches of cells. Red—Phalloidin, shown as 4 (actin cytoskeleton); Blue—DAPI, shown as 3 (nuclear staining); Green, shown as 2 (CD54, CD68, CD45, CD84 respectively).

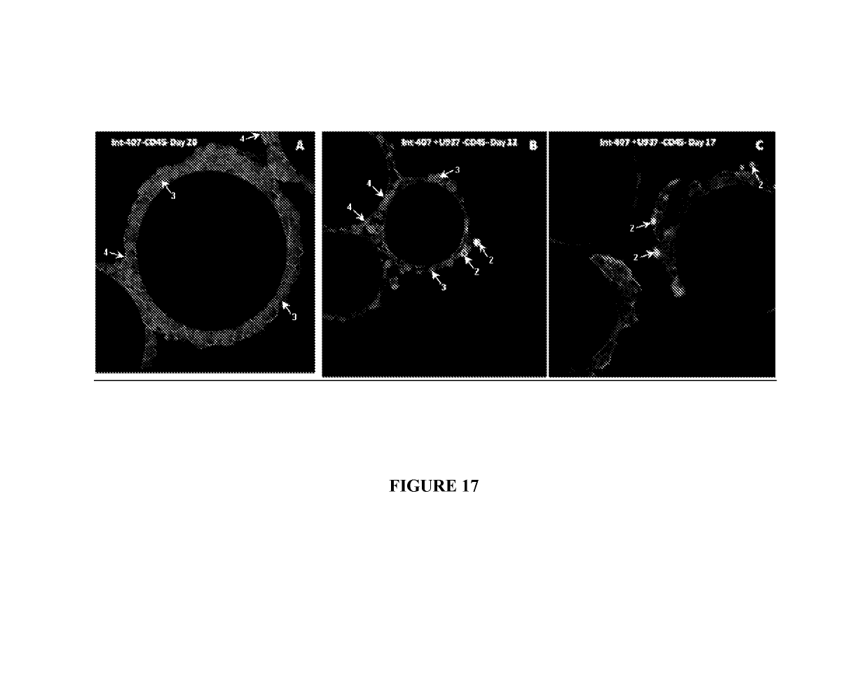

Specific markers were profiled to ensure optimal differentiation of each cell type in the three-dimensional co-culture aggregates, and include: Intestinal cells—ZO-1, MUC5AC; Macrophages—CD54, CD45, CD68, CD84. Markers specifically profiled at this stage of the study include: a) ICAM-1, CD54 expressed both in monocytes and macrophages (more so in the latter) as well as on the apical cell side of the intestinal epithelium co-cultured with differentiated U937 cells; b) CD45, expressed in all mononuclear phagocytic cells but not in epithelial cells; c) CD68, known as microsialin, predominantly expressed in cytoplasmic granules of monocytes/macrophages, dendritic cells, and granulocytes, and d) CD84, a Cd2 subset of the Ig superfamily, expressed on mature B cells, B cell lines, monocytes, and it strongly stains tissue macrophages. Each of these markers was also profiled in the monotypic three-dimensional models of each cell type. Immunohistochemical profiling showed important physiological differences in expression and distribution of these markers between the 3-D co-culture models as compared to 3-D monotypic cultures. Representative immunohistochemical profiling comparisons for select markers between 3-D co-cultures and 3-D monotypic cultures of each cell type, are shown in FIGS. 14 and 15. In developing the Int-407 and U937 co-culture models using this method, initial studies have shown the incorporation of U937 cells using CD45 marker. The Int-407 cells did not appear as healthy; further optimization is contemplated for this particular model (FIG. 17). FIG. 17 shows immunohistochemical profiling of small intestinal (Int-407) epithelial cells and PMA differentiated macrophages (U937) grown as 3-D co-cultures (FIGS. B and C), and 3-D monotypic cultures (FIG. A). Confocal images of cultures from various timepoints were stained with antibodies against phagocytic marker CD45 (FIGS. A, B and C). Red—Phalloidin, shown as 4 (actin cytoskeleton); Blue—DAPI, shown as 3 (nuclear staining); Green, shown as 2 (CD45).

FIG. 15 shows immunohistochemical profiling of colon (HT29) cells and PMA differentiated macrophages (U937) grown as 3-D co-cultures (FIGS. B, D, F, G-L), and 3-D monotypic cultures (FIGS. A, C and E). Confocal images of cultures from various timepoints were stained with antibodies against epithelial differentiation markers MUC5AC (FIGS. A and B), ZO-1 (FIGS. C, D, E, F), and monocyte/macrophage markers CD45 (FIGS. G, I, J, L), and CD54 (FIG. K). Phagocytosis of 2 mm fluorescent beads by the 3-D HT29-U937 (PMA-differentiated) co-culture are shown (FIGS. H-J). Macrophage-like cells in the 3-D co-culture are labeled with the CD45 antibody (orange, shown as 1) (FIGS. G and J) showing specific uptake of beads (green, shown as 2) (FIGS. H-J) and the merging of 3-D HT29-U937co-cultures for CD45 (orange, shown as 1) and beads (green, shown as 2) resulting in yellow, shown as 5, I) further confirming these CD45 positive cells and not the colonic epithelial cells are involved in the phagocytic activity. Uptake of GFP labeled SL1344 bacteria after 1 hour by the CD45 positive macrophage like cells also supports functionality of the co-culture model. Major differences in expression and distribution patterns of these proteins were observed between 3-D aggregates with 3-D co-cultures exhibiting expression and organization relevant to that found in normal tissues. Data are from three experiments and are representative of independent batches of cells. Red—Phalloidin, shown as 4 (actin cytoskeleton); Blue—DAPI, shown as 3 (nuclear staining); Green, shown as 2 (MUC5AC, ZO-1, Beads, SL1344); Orange, shown as 1 (CD45, CD54), Yellow, shown as 5 (CD45+ Green Fluorescent Beads).

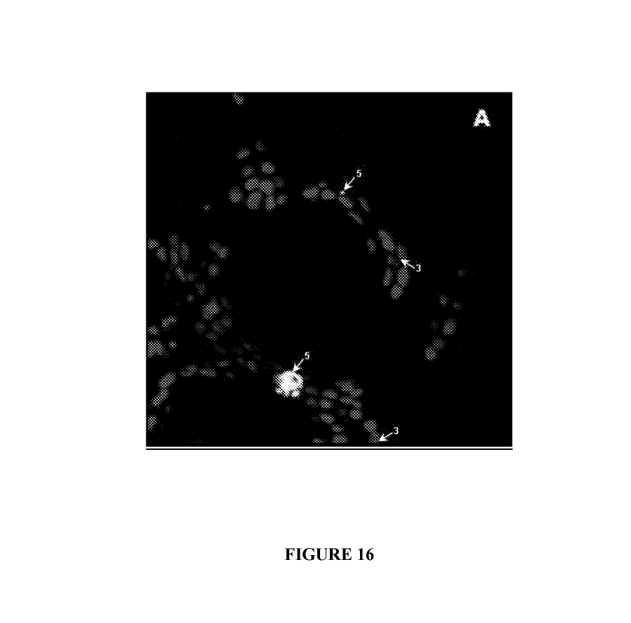

FIG. 16 shows immunohistochemical profiling showing phagocytosis of fluorescent beads by PMA differentiated macrophages (U937) grown as co-cultures with HT29 colonic epithelial cells. Blue (DAPI) is shown as 3 (nuclear staining), and yellow fluorescent beads are shown as 5.

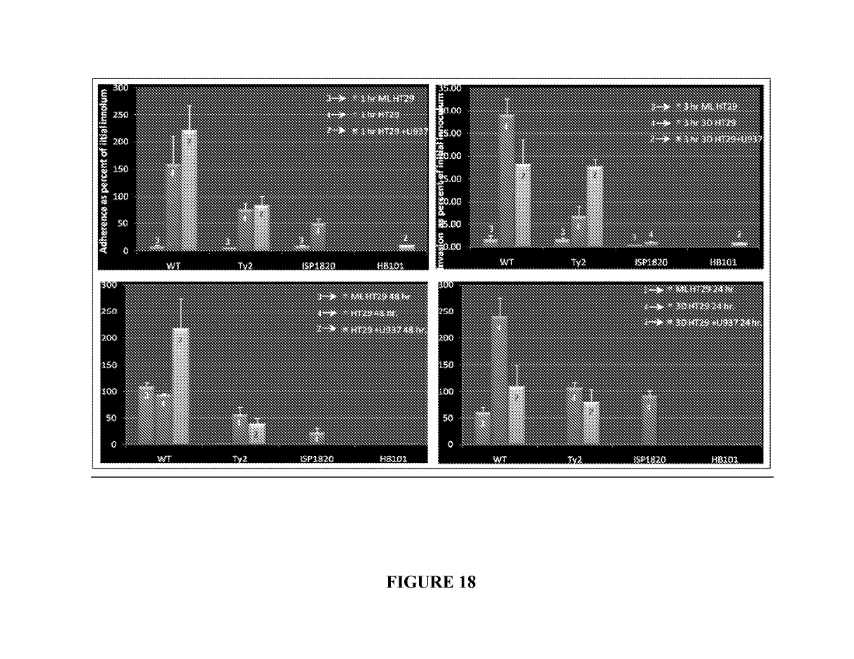

In FIG. 18, this co-culture model was used to examine adherence, invasion and survival of Salmonella Typhimurium and Salmonella Typhi during infection of the host, bacteria such as S. Typhimurium encounters several different host cell types, including the lining of epithelial cells, macrophages and dendritic cells. Initial studies reveal interesting differences between bacterial colonization at the 3 hour and 48 hour time points.

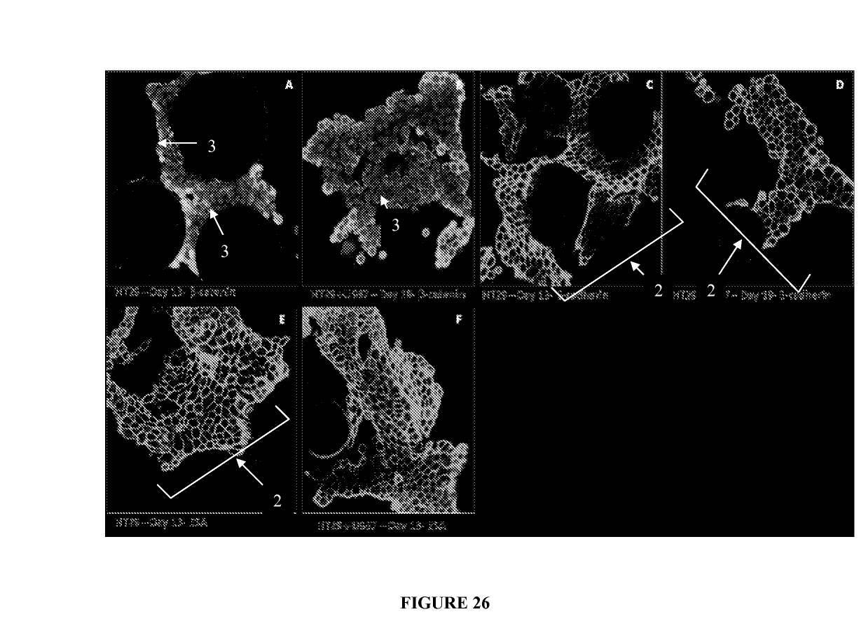

FIGS. 26B, D, and F shows immunohistochemical profiling of colon (HT29) cells and PMA-differentiated macrophages (U937) grown as three-dimensional co-cultures. FIGS. 26A, C, and E show three-dimensional 3-D monotypic cultures. Confocal images of cultures of Day 13 and Day 19 were stained with antibodies against epithelial differentiation markers β-catenin (A, B), E-cadherin (C, D) and Epithelial Specific Antigen (ESA) (E,F), Blue—DAPI (nuclear staining); Green (β-catenin, E-cadherin and ESA). The addition of macrophages to the three-dimensional intestinal epithelial model did not alter the physiological expression pattern of the tight junctional proteins beta catenin and E-cadherin. ESA profiling demonstrated that the macrophages do not express this epithelial cell specific marker, as predicted.

Summary.

Example 2 demonstrated that co-cultures of human colon and monocyte cell lines cultured in the RWV bioreactor aggregate based on natural cellular affinities and self-assemble into biologically relevant three-dimensional aggregates. Example 2 also demonstrated that a single media is compatible with establishment and differentiation of both cell types in the three-dimensional co-culture models. In general, expression patterns of cell specific markers in the three-dimensional co-culture models revealed tissue organization and differentiation relevant to that found in the normal tissue in vivo, as opposed to the same cells grown as monotypic three-dimensional cultures. In addition, the expression of the different macrophage/phagocytic markers validated the incorporation of macrophages in this model, and the uptake of beads and bacteria by this immunocompetent model further validated the functionality. These results demonstrated the physiological relevance of this cell culture system, which is useful as a valuable tool for developing drugs and testing various conditions of pathophysiological relevance associated with the intestine.

### Example 3

### Three-Dimensional Co-Culture of Human Neuronal Cells Cultured with Astrocytes and/or Monocytes

The studies in this Example 3 established and characterized a biologically relevant three-dimensional multicellular co-culture model with human neuronal cells cultured in combination with macrophages and astrocytic cells. Furthermore, this Example identified HIV-associated dementia complex (HAD) molecular markers in the three-dimensional multicellular co-culture model.

Establishment of Multicellular Organotypic Co-Cultures. Cell-Line Derived Model 1:

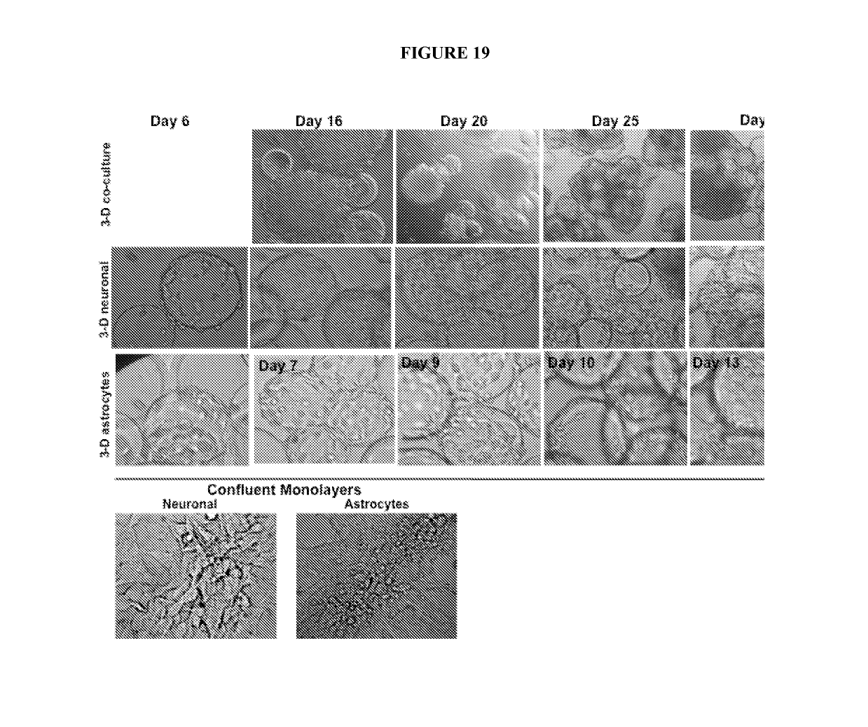

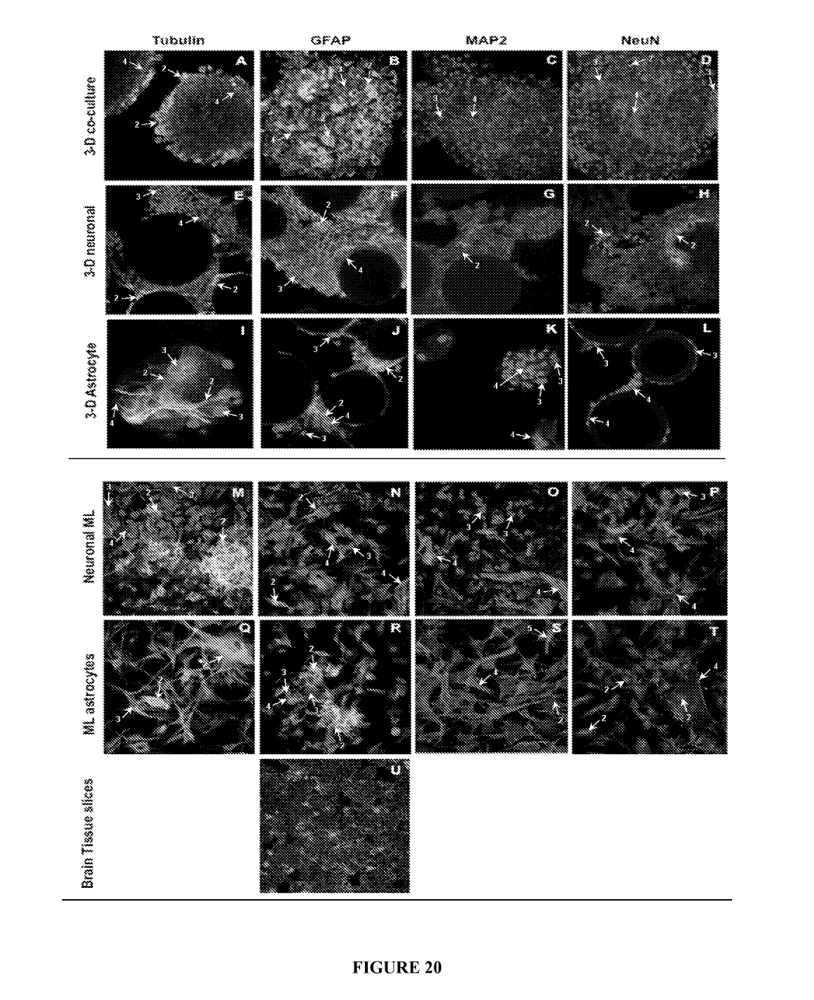

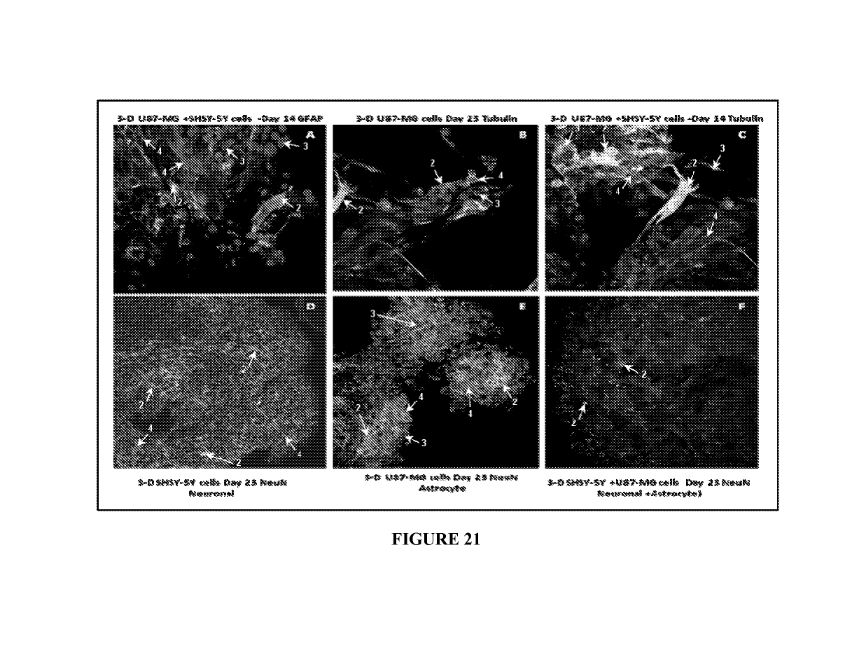

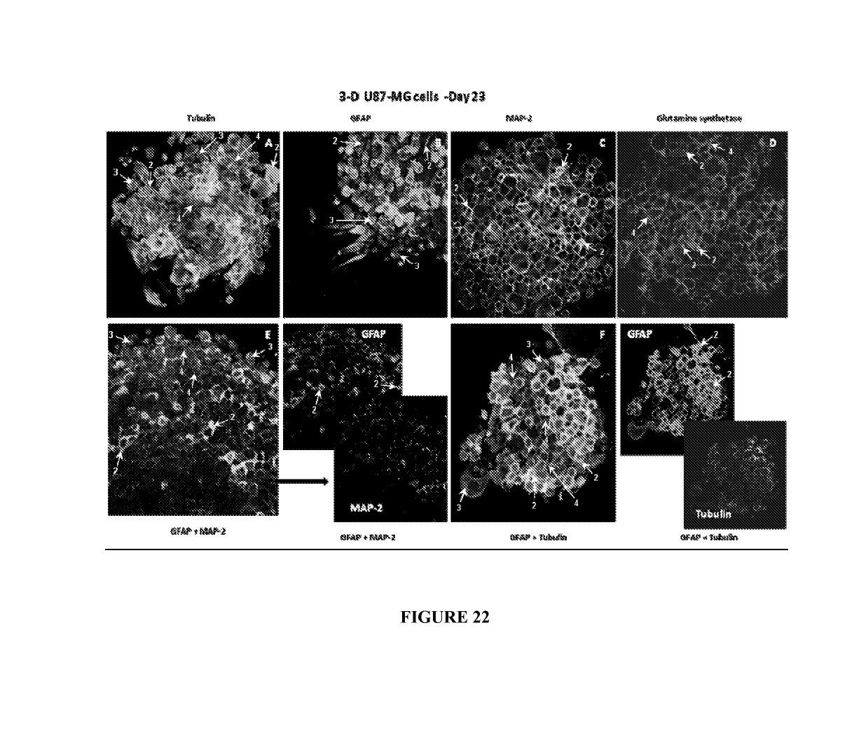

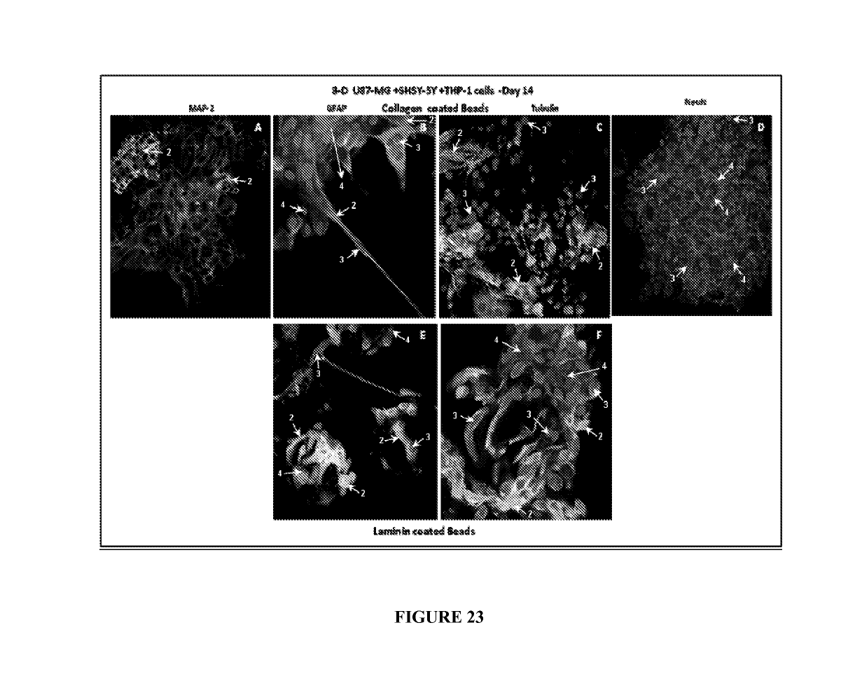

The studies in Example 3 established and optimized three-dimensional multi-cellular co-culture models derived from human cell lines with neuronal cells (SH-SY5Y neuroblastoma) and astrocytes (HTB-14/U87-MG astrocytoma-glioblastoma). Extensive optimization parameters empirically tested and the outcomes determined to date for this three-dimensional co-culture model development are shown in Table 3. Separate 3-D monotypic cultures of each of these cell lines were also established as controls for the multicellular co-culture model. For parameters 1-3 listed in Table 3, sample aliquots of 3-D cultures (monotypic and co-culture models) were removed from the bioreactor at times indicated below and monitored for viability (trypan blue exclusion), kinetics of aggregate formation (light microscopy—FIG. 19), and differentiation for cell-specific markers (confocal microscopy—FIG. 20) throughout the course of growth at the indicated timepoints to determine optimal time for harvesting. In addition, the differentiation for neuronal cells or the astrocytic cells grown alone in the bioreactor or both cells combined were examined using different cell-specific markers (FIGS. 21 and 22). The co-culture model including all three cell lines was also studied using two different ECM coatings on the microcarrier beads (collagen and laminin) to determine which scaffold allowed for better differentiation (FIG. 23).

TABLE 3
Optimization parameters empirically determined for 3-D co-culture model establishment

| Parameter | Conditions tested | Result |
|---|---|---|
| 1) Timing of | Sequential addition - addition of | While addition of neuronal cells |
| addition of | astrocytes at day 9 or 18 to | prior to astrocytes yielded robust 3- |
| different cell types | neuronal cells growing in 3-D on | D aggregate co-culture formation |
| (SH-SY5Y | microcarriers in the RWV | with expression of differentiation |
| neuronal cells and | bioreactor | markers under the conditions tested, |
| HTB-14 astrocytes) | | addition of astrocytes prior to |
| | | neuronal cells is also contemplated, |
| | | as well simultaneous addition of |
| | | both cell types. |
| 2) Ratio of | Ratios tested - 1:1 (neuronal | 1:1 ratio yielded representative 3-D |
| different cell types | cells:astrocytes) | co-culture models that self- |
| (SH-SY5Y | | assembled into aggregates |
| neuronal cells and | | containing both neuronal cells and |
| HTB-14 astrocytes) | | astrocytes, as determined by |
| | | immunohistochemical profiling. |
| | | Flow cytometry will also be |
| | | performed to quantitate cell type |
| | | percentages in the aggregates. |
| 3) Optimal media | Media types tested: | GTSF-2 was compatible for 3-D |
| to use in co-culture | Neuronal: a) EMEM/F12 with and | co-culture model establishment and |
| model | without heat-inactivated serum (to | supported differentiation of both |
| establishment (SH- | control for unacceptable pH | cell types. |
| SY5Y neuronal | differences observed during culture | |
| cells and HTB-14 | of these cells); b) EMEM/F12 with | |
| astrocytes) | and without Pen/Strep; c) | |
| | Advanced DMEM/F12; and d) GTSF-2. | |
| | Astrocytes: a) EMEM and b) GTSF-2. | |
| 4) Differentiation | A) Times profiled via light & | In general, expression patterns of |
| and viability | Confocal microscopy: | cell specific markers in 3-D co- |
| profiling | 3-D neuronal cells: days 6, 12, 15- | culture models revealed tissue |
| | 17, 20, 22, 25-27, 29, 31, and 34. | organization and differentiation |
| | 3-D astrocytes: days 6-13 (later | relevant to that found in the normal |
| | timepoints on-going) | tissue in vivo, as compared to the |
| | 3-D co-cultures (neuronal cells + | same cells grown as monotypic 3-D |
| | astrocytes): days 6, 12, 15-17, 20, | cultures and poor organization and |
| | 22, 25-27, 29, 31, and 34. | abnormal/diffuse expression |
| | B) Differentiation markers tested: | observed in confluent monolayers |
| | Neurons: MAP-2, NeuN, Class III | suggestive of tumor cell de- |
| | b-Tubulin, Synapsin 1, TU-20, and | differentiation. |
| | Nestin; Astrocytes: GFAP, S100, | |
| | Tubulin, and Glutamine synthetase | |

*Note:
All 3-D cell models and monolayers showed >97% viability as profiled by trypan blue.

Model 1 development was completed by refining parameters listed in Table 1: a) timing of addition of different cell types—i.e. addition of astrocytes prior to neuronal cells—and also simultaneous cell addition; b) ratio of different cell types—10:1 ratio of astrocytes to neuronal cells, c) immunohistochemical validation of model differentiation under conditions a-b, including double-staining with cell-specific markers; d) addition of third cell type, the human monocytic cell line, THP-1, to 3-D co-culture model of neuronal cells and astrocytes; e) immunohistochemical and morphological validations.

Validation of the Three-Dimensional Co-Culture Model:

Models were characterized by a) light microscopy for kinetics of 3-D aggregate formation (FIG. 19), and b) immunohistochemical profiling using antibodies against cell-specific markers of differentiation characteristic for each respective cell type in vivo (FIG. 21-23). Specific markers were profiled to ensure optimal differentiation of each cell type in the 3-D co-culture aggregates, and include: Neurons—MAP-2, NeuN, Class III β-Tubulin, Synapsin 1, TU-20, and Nestin; Astrocytes—GFAP, 5100, Tubulin, and Glutamine synthetase, Monocytes/Dendritic cells—CD83 and CD68. Markers specifically profiled at this stage of the study include: a) Microtubule-Associated Protein (MAP-2), an abundant neuronal cytoskeletal protein that binds to tubulin and stabilizes microtubules; b) NeuN (NEUronal Nuclei) a DNA-binding, neuron-specific protein which is present in most CNS and PNS neuronal cell types of all vertebrates tested; c) Glial Fibrillary Acidic Protein (GFAP), a class-III intermediate filament and the main constituent of intermediate filaments in astrocytes and a cell specific marker that distinguishes differentiated astrocytes from other glial cells, and d) Tubulin, one of several members of a small family of globular proteins which make up microtubules. Each of these markers was also profiled in the monotypic 3-D models of each cell type as well as for monolayers of each cell type. In addition, monocyte/dendritic cell specific markers were also profiled and include a) CD68, known as microsialin, is predominantly expressed in cytoplasmic granules of monocytes/macrophages, dendritic cells, and granulocytes, and b) CD83, a heavily glycosylated membrane protein that is expressed in mature dendritic cells. Immunohistochemical profiling showed important physiological differences in expression and distribution of these markers between the 3-D co-culture models as compared to 3-D monotypic cultures and monolayers of each cell. In addition, normal human brain tissue sections from cerebellum and left frontal lobe (Abeam) were profiled for GFAP expression as controls. Representative immunohistochemical profiling comparisons for select markers between 3-D co-cultures, 3-D monotypic cultures and monolayers of each cell type, and normal human brain tissue paraffin sections are shown in FIG. 20. (Note—Confluent monolayers were used for all comparisons). Additional profiling are shown in FIGS. 21-23.

FIG. 19 shows light microscopy images of kinetics of 3-D aggregate formation by neuronal (SH-SY5Y) and astrocyte (HTB-14) cells grown as 3-D co-cultures (first panel), 3-D neuronal cells (second panel) and 3-D astrocytes (third panel). The process of aggregate formation of these cultures was monitored by imaging at particular days using different magnifications. Shown below are images of 3-D cells in co-culture removed from the bioreactor at Days 16, 20, 25 and 27. *(Note: on day 9, the astrocytes were added to the neuronal cells, thus Day 6 is missing for the 3-D co-culture model). The same days were profiled for 3-D monotypic cultures of neuronal cells, and similar days for 3-D monotypic cultures of astrocytes (which were imaged on days 6, 7, 9, 10 and 13). Lower panel shows confluent neuronal and astrocyte monolayer images.

FIGS. 20A-B shows immunohistochemical profiling of neuronal (SH-SY5Y) and astrocyte (HTB-14) cells grown as 3-D co-cultures (A-D), 3-D monotypic cultures (E-F neuronal; I-L astrocytes), or monolayers (M-P neuronal; Q-T astrocytes). Confocal images of cultures from various timepoints were stained with antibodies against Tubulin (A,E,I,M,Q), GFAP (B,F,J,N, R), MAP2 (C,G,K,O,S), and NeuN (D,H,L,P,T). Major differences in expression and distribution patterns of these proteins were observed between 3-D aggregates and monolayers, with 3-D co-cultures exhibiting expression and organization relevant to that found in normal tissues. Data are from two experiments and are representative of independent batches of cells. (U represents normal human brain cerebellum tissue slide stained with anti-GFAP). Red—Phalloidin, shown as 4 (actin cytoskeleton); Blue—DAPI, shown as 3 (nuclear staining); Green, shown as 2 (Tubulin, GFAP, MAP2, or NeuN, respectively).

FIG. 21 shows immunohistochemical profiling of neuronal (SH-SY5Y) and astrocyte (HTB-14/U87-MG) cells grown as 3-D co-cultures (A,C, F), 3-D monotypic cultures (D, neuronal; B, E astrocytes). Confocal images of cultures from various timepoints were stained with antibodies against Tubulin (B, C), GFAP (A), and NeuN (D-F). Major differences in expression and distribution patterns of these proteins were observed in 3-D aggregates, with 3-D co-cultures exhibiting expression and organization relevant to that found in normal tissues. (Red—Phalloidin, shown as 4 (actin cytoskeleton); Blue—DAPI, shown as 3 (nuclear staining); Green, shown as 2 (Tubulin, GFAP, or NeuN).

FIG. 22 shows immunohistochemical profiling of astrocyte (HTB-14/U87-MG) cells grown as 3-D aggregates. Confocal images of cultures from Day 23 were stained with antibodies against Tubulin (A), GFAP (B), MAP-2 (C) Glutamine synthetase (D) and dual staining with GFAP and MAP-2 (E) and GFAP and Tubulin. Major differences in expression and distribution patterns of these proteins were observed in 3-D astrocyte aggregates, with cultures exhibiting expression and organization relevant to that found in normal tissues. (Red—Phalloidin, shown as 4 (actin cytoskeleton); Blue—DAPI, shown as 3 (nuclear staining); Green, shown as 2 (Tubulin, GFAP, MAP-2, or Glutamine sythetase).

FIG. 23 shows immunohistochemical profiling of neuronal (SH-SY5Y) astrocyte (HTB-14) and monocytic cells (THP-1) grown as 3-D co-cultures (A-F) in a collagen coated ECM vs. laminin coated ECM on microcarrier beads. Confocal images of cultures from Day 14 were stained with antibodies against MAP-2 (A), GFAP (B and E), Tubulin (C and F), and NeuN (D). Major differences in expression and distribution patterns of these proteins were observed with these 3-D co-cultures exhibiting expression and organization relevant to that found in normal tissues. Red—Phalloidin, shown as 4 (actin cytoskeleton); Blue—DAPI, shown as 3 (nuclear staining); Green, shown as 2 (MAP2, GFAP, Tubulin, or NeuN, respectively).

Summary.

Example 3 demonstrated that co-cultures of human neuronal, astrocyte and monocytic cell lines cultured in the RWV bioreactor aggregate based on natural cellular affinities and self-assemble into biologically relevant three-dimensional aggregates. This Example also demonstrated that a single media is compatible with establishment and differentiation of both cell types in the 3-D co-culture models. In general, expression patterns of cell specific markers in the 3-D co-culture models revealed tissue organization and differentiation relevant to that found in the normal tissue in vivo, as compared to the same cells grown as monotypic 3-D cultures and the poor organization and abnormal/diffuse expression observed in monolayers suggestive of tumor cell de-differentiation. These results demonstrate the physiological relevance of this cell culture system for model development and application toward, for example, HIV infection studies.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention.

1. A method of producing a three-dimensional, physiologically relevant immune tissue system, the method comprising:
a) introducing an immune cell and at least one other cell type into a low fluid shear environment; and
b) co-culturing the immune cell and the at least one other cell type under conditions selected to produce a three-dimensional immune tissue system with one or more physiologically relevant characteristics.

2. The method of claim 1, wherein the one or more physiologically relevant characteristics are selected from the group consisting of one or more differentiated and functional cells, assembly into relevant three-dimensional aggregates, production of extracellular matrix components, and physiologically relevant cell type ratios.

3. The method of claim 1, wherein the immune cells are selected from the group consisting of monocytes, astrocytes, neuronal cells, macrophages, dendritic cells, B cells, T cells, natural killer cells, basophils, eosinophils, and neutrophils from healthy and/or diseased subjects.

4. The method of claim 3, wherein the immune cells are astrocytes and neuronal cells.

5. The method of claim 4, wherein the immune cells further comprise monocytes.

6. The method of claim 1, further comprising culturing the immune cell and/or the at least one other cell type in a monolayer before placing in the low fluid shear environment.

7. The method of claim 1, further comprising developing the immune cell and/or at least one other cell type into three-dimensional cells before placing in the low fluid shear environment.

8. The method of claim 1, comprising first placing the immune cell in the low fluid shear environment, first placing the at least one other cell type in the low fluid shear environment, or placing the immune cell and the at least one other cell type simultaneously in the low fluid shear environment.

9. The method of claim 1, comprising developing the cells into three-dimensional cells on a scaffold.

10. The method of claim 9, wherein the scaffold is made of microcarrier beads.

11. The method of claim 9, wherein the at least one other cell type is an immune or epithelial cell.

12. The method of claim 11, wherein the one or more physiologically relevant characteristics are selected from the group consisting of a differentiated epithelium, one or more functional macrophage-like cells, a localization of macrophage-like cells on or beneath the epithelial surface, production of one or more extracellular matrix components, and a physiologically relevant macrophage-to-epithelial cell ratio.

13. The method of claim 12, wherein the physiologically relevant macrophage-to-epithelial cell ratio ranges from about 1:30 to about 1:40.

14. The method of claim 11, wherein the epithelial cells are selected from the group consisting of alveolar, bronchial, small intestinal, large intestinal, cervical, urogenital, gastrointestinal tract, respiratory tract, and vaginal epithelial cells from healthy and/or diseased subjects.

15. The method of claim 11, wherein the epithelial cells and the immune cells are derived from human cell lines.

16. The method of claim 11, wherein the epithelial cells are small intestinal epithelial cells, and the immune cells are monocytes.

17. The method of claim 11, wherein the epithelial cells are large intestinal epithelial cells, and the immune cells are monocytes.

18. The method of claim 11, wherein the immune cell is a monocyte, and the at least one other cell type is an alveolar epithelial cell.

19. The method of claim 18, further comprising culturing the alveolar epithelial cells in a monolayer and developing the alveolar epithelial cells into three-dimensional cells in the low fluid shear environment.

20. The method of claim 19, wherein the ratio of the monocytes to the three-dimensional alveolar epithelial cells ranges from about 1:100 to about 100:1.

21. The method of claim 1, wherein the low fluid shear environment ranges from about 0 dynes/cm2 to about 10.0 dynes/cm.2

22. The method of claim 1, wherein the time period in the low fluid shear environment ranges from about 1 day to about 40 days.

23. The method of claim 1, wherein the low fluid shear environment is provided by one or more bioreactors.

24. The method of claim 23, wherein the bioreactor is a rotating wall vessel (RWV).

25. The method of claim 24, wherein the bioreactor has a rotation speed that ranges from about 10 rpm to about 30 rpm.

26. The method of claim 23, wherein the RWV is a slow transfer/turning lateral vessel (STLV).

27. The method of claim 23, wherein the RWV is a high-aspect rotating vessel (HARV).

28. The method of claim 1, wherein the low fluid shear environment is a spaceflight environment.

29. The method of claim 1, wherein the conditions appropriate for producing a three-dimensional immune tissue system with physiologically relevant characteristics are selected from the group consisting of appropriate culture medium, temperature, pH, oxygen levels, composition of the extracellular matrix, and time in the low fluid shear environment.

30. The method of claim 29, wherein the culture medium is GTSF-2.

31. The method of claim 30, wherein the time in the low fluid shear environment ranges from about 24 hours to about 1 year.

32. The method of claim 1, further comprising conducting one or more biochemical analyses to determine that the three-dimensional tissue system has one or more physiologically relevant characteristics.

33. A kit for producing a three-dimensional, physiologically relevant tissue system, comprising an immune cell line and at least one other cell line; and informational material for producing a three-dimensional, physiologically relevant tissue system.
